(19) United States
(12) Patent Application Publication
(10) Pub. No.: US 2011/0081677 A1
(43) Pub. Date: Apr. 7, 2011

(54) Active Microfluidic Membranes

(75) Inventors: Xiaolong Luo, Adelphi, MD (US); Gary W. Rubloff, Clarksville, MD (US); Dean L. Berlin, College Park, MD (US); William E. Bentley, Annapolis, MD (US); Susan Buckhout-White, Silver Spring, MD (US); Yi Cheng, Beltsville, MD (US); Jordan Betz, Columbia, MD (US)

(73) Assignee: University of Maryland, College Park, College Park, MD (US)

(21) Appl. No.: 12/890,017

(22) Filed: Sep. 24, 2010

Related U.S. Application Data
(60) Provisional application No. 61/247,341, filed on Sep. 30, 2009.

(51) Int. Cl. C12M 1/00; C08B 37/04; C08B 37/08; C12N 11/10; C07K 1/04; C12P 1/00; B01L 3/00

(52) U.S. Cl. 435/41; 435/283.1; 435/289.1; 536/3; 536/20; 435/178; 530/402; 422/502

(57) ABSTRACT

The present invention relates to a biofabricated Active Microfluidic Membrane (AMM) in a microfluidic network of a microfluidic device and a method for the in situ biofabrication of such a microfluidic network. More specifically, the invention relates to devices exhibiting (and methods of) positioning (i.e., erecting, modifying or removing a membrane matrix in situ in a microchannel of a microfluidic network of a microfluidic device. In one embodiment, the membrane comprises a single type of matrix constituent, such as chitosan, alginate, etc. Alternatively, the membrane may be composed of two or more matrix constituents, which may be integrated into one another or layered adjacent to one another.

### CROSS-REFERENCE TO RELATED APPLICATIONS

This application claims priority to U.S. Patent Application Ser. No. 61/247,341 (filed Sep. 30, 2009, pending), which application is herein incorporated by reference in its entirety.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of NSF SCO35224414 awarded by the National Science Foundation.

### BACKGROUND OF THE INVENTION

1. Field of the Invention

The present invention relates to a biofabricated Active Microfluidic Membrane (AMM) in a microfluidic network of a microfluidic device and a method for the in situ biofabrication of such a microfluidic network. More specifically, the invention relates to devices exhibiting (and methods of) positioning (i.e., erecting, modifying or removing a membrane matrix in situ in a microchannel of a microfluidic network of a microfluidic device. In one embodiment, the membrane comprises a single type of matrix constituent, such as chitosan, alginate, etc. Alternatively, the membrane may be composed of two or more matrix constituents, which may be integrated into one another or layered adjacent to one another.

2. Description of Related Art

Microfluidic networks include microscopically defined channels, pathways and/or components that manipulate fluids on the scale of microliters or nanoliters. An array of functional units (e.g., valves, pumps, reaction chambers, channels, etc.) may be incorporated onto a single chip to create a “lab-on-a-chip” (“LOC”). A common application for such networks is to provide precise control and experimentation on biochemical processes. For example, enzymes are well-understood catalysts that have high specificity and known reaction rates. By immobilizing enzymes in active form, they may be assembled into engineered microfluidic networks. However, the capability to efficiently immobilize catalytically active enzymes in a microfluidic network remains challenging.

Enzymes may be immobilized in microchannels using either physical entrapment such as packed beads or surface immobilization onto the wall surfaces of a microchannel (Bilitewski, U. et al. (2003) “Biochemical analysis with microfluidic systems,” Analytical and Bioanalytical Chemistry 377:556-569; Hickey, A M et al. (2007) “Immobilization and thermophilic enzymes in miniaturized flow reactors,” Biochem. Soc. Trans. 35:1621-1623; Krenkova, J. et al. (2004) “Immobilized microfluidic enzymatic reactors,” Electrophoresis 25:3550-3563). In the scheme of packed beads in microchannels, effort is needed to confine the beads in the microfluidic network (Ku, B S et al. (2006) “Chip-based polyketide biosynthesis and functionalization,” Biotechnology Progress 22:1102-1107). In the scheme of surface immobilization, the proximity of reaction substrates to the immobilized enzyme and the departure of reaction products are mostly transported by passive diffusion of small molecules, rather than the active convection in the flow stream (Hu, G Q et al. (2007) “Modeling micropatterned antigen-antibody binding kinetics in a microfluidic chip,” Biosens. Bioelectron. 22:1403-1409). Thus, the conversion efficiency in conventional techniques is limited given much of the introduced substrate passes the enzyme site un-reacted.

In multi-step biochemical reactions, spatially separating individual reaction steps in microfluidics allows for a better understanding of reaction details and testing of molecules that can modify pathways and kinetics (Logan, T C et al. (2007) “Photopatterning enzymes on polymer monoliths in microfluidic devices for steady-state kinetic analysis and spatially separated multi-enzyme reactions,” Analytical Chemistry 79:6592-6598). Therefore, it would be desirable to immobilize the enzymes at specific sites in the microfluidic network. It has been demonstrated that metabolic pathway enzymes can be sequentially assembled in microfluidic devices, and used to simulate biologically relevant processes (Luo, X et al. (2008) “Programmable assembly of a metabolic pathway enzyme in a pre-packaged reusable bioMEMS device,” Lab on a Chip 8:420-430). However, the enzymatic conversion efficiency of such devices is subject to the limitations associated with having the enzyme immobilized onto the microchannel surface. In particular, enzyme located at the side of a flow channel is a geometry that prevents transport of substrate species to the enzyme, thus reducing efficiency of substrate-enzyme interaction. Other geometries, such as immobilizing the enzyme on a porous membrane such that transport of species must flow through the membrane, would offer improved substrate-enzyme interactions and higher enzymatic conversion efficiency.

The integration of membrane functionality into microfluidics has attracted substantial attention. Mass transport control is achieved by integrated membranes for applications such as filtration (Noblitt, S. D. et al. (2007) “Integrated membrane filters for minimizing hydrodynamic flow and filtering in microfluidic devices,” Analytical Chemistry, 79(16):6249-6254; Long, Z. et al. (2006) “Integration of nanoporous membranes for sample filtration/preconcentration in microchip electrophoresis,” Electrophoresis, 27(24):4927-4934; Thorslund, S. et al. (2006) “A hybrid poly(dimethylsiloxane) microsystem for onchip whole blood filtration optimized for steroid screening,” Biomedical Microdevices, 8(1):73-79; Hsieh, Y. C. et al. (2007) “On-chip microdialysis system with flow-through sensing components,” Biosens. Bioelectron., 22(11):2422-2428; Braschler, T. et al. (2005) “Gentle cell trapping and release on a microfluidic chip by in situ alginate hydrogel formation,” Lab on a Chip 5:553-559), microdialysis (Kurita, R. et al. (2005) “Miniaturized one-chip electrochemical sensing device integrated with a dialysis membrane and double thin-layer flow channels for measuring blood samples,” Biosensors and Bioelectronics, 21(8):1649-1653; Hsieh, Y. C. et al. (2005) “Glucose recovery in a microfluidic microdialysis biochip,” Sensors and Actuator B: Chemical, 107(2):649-656), extraction (Cai, Z. X. et al. (2006) “A microfluidic chip based liquid-liquid extraction system with microporous membrane,” Analytica Chimica Acta, 556(1):151-156), and gas-liquid exchange (Lange, D. et al. (2005) “A microfluidic shadow imaging system for the study of the nematode Caenorhabditis elegans in space,” Sensors and Actuators B: Chimcal, 107(2):904-914). Approaches for membrane integration include direct incorporation of commercial membranes or forming membranes as part of the bioMEMS chip fabrication process, both of which pose difficulty in packaging the microfluidic chips, or require additional complexity and cost in fabrication (de Jong, J et al. (2006) “Membranes and microfluidics: a review,” Lab on a Chip, 6:1125-1139).

In situ photopolymerization and thermo-gelation have been investigated to form porous structures in microchannels (Moorthy, J et al. (2003) “In Situ fabricated porous filters for microsystems,” Lab on a Chip, 3:62-66; Tan, W. et al. (2003) “Microfluidic Patterning of Cellular Biopolymer Matrices for Biomimetic 3-D Structures,” Biomedical Microdevices, 5(3):235-244). For example, membrane-like hydrogels produced by ultraviolet photopolymerization (Albrecht, D. R. et al. (2006) “Probing the role of multicellular organization in three-dimensional microenvironments,” Nature Methods, 3(5):369-375; Chee Ping N. et al. (2008) “A perfusable 3D cell-matrix tissue culture chamber for in situ evaluation of nanoparticle vehicle penetration and transport,” Biotechnology and Bioengineering, 99(6):1490-1501) or thermo-sensitive gelation (Ling, Y et al. (2007) “A cell-laden microfluidic hydrogel,” Lab on a Chip, 7:756-762; Shibata, K. et al. (2008) “Collagen micro-flow channels as an for in vitro blood-brain barrier model,” Japanese Journal of Applied Physics, 47(6, Pt.2):5208-5211; Sundararaghavan, H. G. et al. (2009) “Neurite growth in 3D collagen gels with gradients of mechanical properties,” Biotechnology and Bioengineering, 102(2):632-643) in microfluidics have emerged for creating three dimensional cell culture environments.

However, in many cases, the ultraviolet photopolymerization and thermo-initiative gelation are cytotoxic (Tan W. et al., supra, Biomedical Microdevices, 5(3):235-244); Albrecht, D. R. et al., supra, Nature Methods, 3(5):369-375; Chee Ping N. et al., supra, Biotechnology and Bioengineering, 99(6):1490-1501; Ling, Y et al., supra, Lab on a Chip, 7:756-762; Shibata, K. et al., supra, Japanese Journal of Applied Physics, 47(6, Pt.2):5208-5211; Sundararaghavan, H. G. et al., supra, Biotechnology and Bioengineering, 102(2):632-643). Moreover, the composition and properties of animal-derived collagen by thermo-gelation has been difficult to control (Tan W. et al., supra, Biomedical Microdevices, 5(3):235-244); Shibata, K. et al., supra, Japanese Journal of Applied Physics, 47(6, Pt.2):5208-5211; Sundararaghavan, H. G. et al., supra, Biotechnology and Bioengineering, 102(2):632-643).

Laminar flow patterning in microfluidics for in situ microfabrication has been exploited for fabrication of polymer membranes in microfluidic devices (Kenis P. J. A. et al. (1999) “Microfabrication inside capillaries using multiphase laminar flow patterning,” Science 285(5424):83-85; Kenis, P. J. A. et al. (2000) “Fabrication inside Microchannels Using Fluid Flow,” Accounts of Chemical Research 33(12):841-847; Hisamoto H. et al. (2003) “Chemicofunctional membrane for integrated chemical processes on a microchip,” Analytical Chemistry 75(2):350-354; Uozumi, Y. et al. (2006) “Instantaneous carbon-carbon bond formation using a microchannel reactor with a catalytic membrane,” Journal of the American Chemical Society 128(50):15994-15995; Orhan, J. B. et al. (2008) “In Situ fabrication of a poly-acrylamide membrane in a microfluidic channel,” Microelectronic Engineering 85(5-6):1083-1085; see also Zhao, B. et al. (2002) “Control and Applications of Immiscible Liquids in Microchannels,” 124(19):5284-5285). Conventional polymer membranes in microfluidics are typically made of non-biological materials, or they are fabricated via non-biological routes. In the case of in situ membrane microfabrication, the lingering initiators and monomer residues from either photopolymerization or polymer chain reactions may be toxic to subsequent biological applications, and subsequent modification of the formed membrane is required for biomolecule assembly (Hisamoto, H. et al., supra, Analytical Chemistry, 75(2):350-354).

Despite all such advances, there remains a need for a unique microfluidic device that enables high enzymatic conversion in the microfluidic network, that is fabricated via a natural process using biological or biocompatible materials, and that does not increase device complexity. The present invention is directed to this and other needs.

### SUMMARY OF THE INVENTION

The present invention relates to the in situ biofabrication of an Active Microfluidic Membrane (“AMM”) in a microfluidic network. More specifically, the invention relates to devices exhibiting (and methods of) positioning (i.e., erecting, modifying or removing a membrane matrix in situ in a microchannel of a microfluidic network of a microfluidic device. In one embodiment, the membrane comprises a single type of matrix constituent, such as chitosan, alginate, etc. Alternatively, the membrane may be composed of multiple matrix constituents, which may be integrated into one another or layered adjacent to one another. Other substituents capable of being biofabricated into an AMM may alternatively or additionally be employed in the disclosed method.

According to one embodiment, the invention provides a method of in situ biofabrication of a freestanding chitosan membrane in a sealed microfluidic device. In a preferred sub-embodiment, the chitosan membrane is provided by tuning the pH gradient at the interface of two laminar flow streams in microfluidics. The biofabricated chitosan membrane may be formed from the biopolymer chitosan via a natural process, which does not need an initiator as in a polymer chain reaction. The formation process of the chitosan membrane is controllable to allow for real-time fabrication with controllable thickness and permeability. Further, the formation process is versatile, allowing for complex intersecting membranes, such as T-shaped interfaces and sequential membrane interfaces as well as for simple membranes.

Further, the present invention provides for the removal of the AMM in a sealed microfluidic device, preferably using acid-base dissolution. The dissolution process is controllable for real-time membrane thinning and/or removal. The dissolution process thus allows for reusability of the device.

The present invention also relates to the enzymatic functionalization of permeable AMMs in a sealed microfluidic device, preferably using conjugation chemistry with catalytic enzymatic components. Such in situ functionalization converts the membrane into an active element in the microfluidic device. The enzyme immobilization is programmable via chemical activation of the pro-tag of enzymes.

The present invention provides the ability to fabricate microfluidic reactors having high enzymatic activity by permitting the direct interaction of the substrate species in the perfusion flow with the enzyme on the permeable AMM to obtain product species. The membrane will preferably be semi-permeable to enzymatic substrate/product, allowing the fluidic streams to either flow through or flow by the membrane. This provides a microfluidic reactor having dramatically enhanced enzymatic conversion efficiency.

The present invention further relates to the formation of an enzymatically active AMM network in a microfluidic device. The membrane may be uniquely functionalized with only one enzymatic component, or multiply functionalized with more than one enzymatic component. Functionalization with multiple enzymatic components may be elegantly employed when engineering devices designed to investigate or mediate multi-step metabolic pathways.

The present invention further relates to a method of purifying a bio-species (particularly a protein) from, for example, a cell extract, etc.) using a sealed or ported microfluidic device having an AMM. In a preferred embodiment, the AMM will be a chitosan membrane, and a protein having an activated pro-tag will be covalently conjugated to the amine groups of the chitosan. The protein-chitosan conjugate may be eluted by mild acid dissolution.

Further, the present invention provides for a lab-on-a-chip process of enzymatic functionalization on AMM to scale up the multiple steps in chemical engineering. The traditional protein purification, storage and spatial protein immobilization are integrated into a one-step enzymatic functionalization on AMM for further enzyme assay.

In detail, the invention concerns a microfluidic device, comprising:
(A) a support including a microchannel defining a first flow path and a second flow path; and
(B) a membrane disposed between the first flow path and the second flow path, the membrane positionable in situ from the micro channel.

The invention particularly concerns the embodiment of such a microfluidic device wherein the membrane comprises a matrix comprising chitosan and/or alginate.

The invention further concerns the embodiments of such microfluidic devices wherein the membrane is semi-permeable and selectively filters a component of one of the first and second flow paths, wherein the membrane is permeable to aqueous solutions or wherein the membrane is permeable to particles smaller than a given (i.e. user selected) size and impermeable to particles greater than the given size.

The invention further concerns the embodiments of such microfluidic devices wherein the membrane includes a first portion and a second portion, the second portion being substantially perpendicular or angularly disposed relative to the first portion.

The invention further concerns the embodiments of such microfluidic devices wherein the microchannel comprises a central portion, and first and second inlet portions in fluid communication with the central portion, the first and second inlet portions converging at the central portion.

The invention further concerns the embodiments of such microfluidic devices wherein the microchannel further comprises first and second outlet portions in fluid communication with the central portion, the first and second outlet portions diverging from the central portion.

The invention further concerns the embodiments of such microfluidic devices that further comprise a conjugated bio-species (e.g., a protein (e.g., an enzymatic component (e.g., an enzyme, enzymatic substrate, or an enzymatic co-factor), a hormone, a receptor, an antibody or antigen-binding fragment thereof, a receptor ligand, etc.), a substrate of an enzymatic reaction, a co-factor, a nucleic acid, a microorganism (e.g., a virus, bacteria, cell (including a mammalian and a human cell, etc.)), or a sub-cellular component thereof (and especially a protein, nucleic acid or a virus) immobilized on the membrane.

The invention further concerns the embodiments of such microfluidic devices that further comprise an enzymatic component immobilized on the membrane to form a catalytically active membrane serving as an enzymatic reaction site for substrate flowing through or flowing by the membrane.

The invention additionally concerns a method of fabricating an Active Microfluidic Membrane (AMM) in a microfluidic device, comprising the steps of:
(A) providing a support defining a sealed microchannel;
(B) generating a fluidic interface between first and second laminar flows within the microchannel; and
(C) fabricating a membrane in situ at the fluidic interface.

The invention further concerns the embodiment of such a method wherein the first laminar flow has a first pH and the second laminar flow has a second pH, thereby creating a pH gradient at the fluidic interface during the generating step.

The invention further concerns the embodiments of such methods wherein the fabricating step comprises tuning the pH gradient between the first and second laminar flows or wherein the fabricating step comprising fabricating a membrane that comprises a matrix of chitosan and/or alginate.

The invention further concerns the embodiments of such methods that comprises the further step of conjugating a bio-species (e.g., a protein (e.g., an enzymatic component (e.g., an enzyme, enzymatic substrate, or an enzymatic co-factor), a hormone, a receptor, an antibody or antigen-binding fragment thereof, a receptor ligand, etc.), a substrate of an enzymatic reaction, a co-factor, a nucleic acid, a microorganism (e.g., a virus, bacteria, cell (including a mammalian and a human cell, etc.)), or a sub-cellular component thereof (and especially a protein, nucleic acid or a virus) onto the membrane.

The invention further concerns the embodiments of such methods that comprise the further step of enzymatically reacting a substrate flowing through or flowing by the membrane.

The invention further concerns the embodiments of such methods that comprise the further step of dissolving in situ at least a portion of the membrane after the fabricating step.

The invention further concerns the embodiments of such methods that comprise the further steps of:
(A) maintaining a first membrane portion after the dissolving step;
(B) altering the first and second laminar flows relative to the first membrane portion, thereby generating a secondary fluidic interface between the altered first and second laminar flows; and
(C) fabricating in situ a second membrane portion at the secondary fluidic interface.

The invention particularly concerns the embodiments of such methods wherein the first membrane portion is angularly disposed relative to the second membrane portion.

The invention further concerns the embodiments of such methods wherein the first laminar flow comprises an acidic chitosan solution, and the second laminar flow comprises a basic solution.

The invention additionally concerns a method of fabricating in situ a free-standing chitosan membrane in a sealed microfluidic device by tuning pH gradient at an interface of adjacent acidic and basic laminar flows within the microfluidic device.

The invention further concerns the embodiment of such method that includes the further step of dissolving in situ at least a portion of the fabricated chitosan membrane using an acidic laminar flow.

### BRIEF DESCRIPTION OF THE DRAWINGS

FIG. 1 illustrates the molecular structure of chitosan and shows its pH responsive solubility.

FIG. 2 shows a microchannel of a microfluidic device and illustrates a pH gradient between a buffer solution and a chitosan solution, and shows a perspective view of a membrane formed thereby within the microchannel. The membrane is shown as a hash-marked rectangle. The pH gradient is depicted as a small rectangle (shown perpendicular to the membrane) and as an expanded gradient rectangle to the left of the microchannel.

FIG. 3 is a schematic diagram of an exemplary pneumatic pumping manifold used to generate and control a stable fluidic interface in a microfluidic network.

FIG. 4 illustrates a flow interface within a microchannel of a microfluidic network.

FIG. 5 is a schematic diagram of a chitosan membrane separating different flow streams within a microchannel.

FIG. 6 is a schematic diagram of a chitosan membrane allowing fluid to permeate through the membrane between different flow streams within a micro channel.

FIG. 7 is a schematic diagram of a chitosan membrane sequestering cells within a microchannel, and the subsequent dissolution of the chitosan membrane to allow the interaction of the different flow streams.

FIG. 8 illustrates a schematic diagram of a chitosan membrane functioning as a filter within a microchannel.

FIG. 9 illustrates a schematic diagram of a chitosan membrane selectively filtering particles from a flow stream within a microchannel.

FIG. 10 illustrates schematic diagram of a chitosan membrane functionalized with particles that aid in visualization or measurement.

FIG. 11 illustrates a schematic diagram of a chitosan membrane functionalized with an enzyme component.

FIG. 12 illustrates a measurable conformal change in a chitosan membrane due to increased enzyme bioactivity.

FIG. 13 illustrates a schematic diagram of enzymatic functionalization and activity on a semi-permeable AMM.

FIGS. 14A-14D illustrate a schematic diagram of an enzyme assembly on a chitosan membrane within a microchannel (FIG. 14A). FIG. 14B illustrates a schematic diagram of the enzyme assembly of FIG. 14A and showing buffer rising. FIG. 14C illustrates a schematic diagram of a catalytically active membrane serving as an enzymatic reaction site as substrate flows through the semi-permeable membrane. FIG. 14D illustrates a schematic diagram of a catalytically active membrane serving as an enzymatic reaction site as substrate flows by the membrane.

FIG. 15 illustrates a schematic diagram of membranes formed in series within a network of microchannels.

FIG. 16 illustrates a schematic diagram of membranes functionalized with multiple enzymatic components.

FIG. 17 shows the synthesis of Autoinducer-2 (AI-2) from S-adenosylhomocysteine (SAH) via enzymatic reaction of Pfs (S-adenosylhomocysteine nucleosidase) and LuxS.

FIG. 18 illustrates a schematic diagram of membranes functionalized for enzymatic reactions in series on the membranes.

FIG. 19 illustrates a schematic diagram of membrane structures in a microfluidics network for implementing protein purification (illustrated with respect to a free-standing chitosan membrane (“FSCM”)).

FIG. 20 illustrates another exemplary pumping strategy to produce a stable flow interface and pH gradient. The lower rectangle of the Figure is a photograph that provides an expanded view of the intersection of the microchannels. The photograph is enhanced to more clearly depict the location of the membrane (thin line, center) and the walls of the microchannel (thick lines).

FIG. 21 illustrates exploded views of a portion of the microchannel in the microfluidic network of the pumping strategy of FIG. 20. The middle and lower rectangles of the Figure are photographs that provide an expanded view of the intersection of the microchannels. The middle rectangle photographs is enhanced to more clearly depict the location of the membrane (solid line, center) and the walls of the microchannel (dotted lines). The small rectangle within the middle rectangle depicts the pH gradient, and is shown in expanded form in the lower rectangle. A color image of the lower rectangle would show a blue line (denoting the response of the pH indicator to a pH of 10) at the upper boundary of the membrane (broad band horizontally traversing the image) with the membrane appearing red (denoting the response of the pH indicator to a pH of 4).

FIGS. 22A-22D show fluorescent microscopy images of a microchannel and the formation of a chitosan membrane. In FIG. 22A, the membrane (shown as a bright white interface extending rightward from the left edge of the microchannel intersection to the center of the image. In FIG. 22B, the membrane has achieved greater length and has nearly reached the right edge of the microchannel intersection. In FIG. 22C, the membrane has fully formed and extends from the left edge of microchannel intersection to the right edge of the microchannel intersection. FIG. 22D shows an optical microscopy image of the microchannel of FIG. 22C and shows the formed chitosan membrane. The images shown in FIGS. 22A-22C have been enhanced to more clearly depict the location of the walls of the microchannel (dotted lines).

FIG. 23 is an image of a membrane showing the microstructure thereof.

FIG. 24 shows images of a membrane showing the microstructure thereof. The “Side View” is an enlarged photograph of the side view of the membrane and indicates that the membrane has a height of 85 μm. The “Top View” is an enlarged photograph of the top view of the membrane and indicates that the membrane has a width of 40 μm.

FIGS. 25A-25D show fluorescent microscopy images of a microchannel and the formation of a T-shaped chitosan membrane. In FIG. 25A, the membrane (shown as a bright white “cross”) extends from the left to right edges of the microchannel intersection and from the top to bottom edges of the microchannel intersection. In FIG. 25B, the portion of the membrane extending from the center of the “cross” to the right edge of the microchannel intersection has decreased in length and no longer reaches the right edge of the microchannel intersection. In FIG. 25C, the portion of the membrane that had initially extended from the center of the “cross” to the right edge of the microchannel intersection has been removed to thereby open a flowable channel between the lower right and upper right microchannels of the microchannel intersection. FIG. 25D shows an optical microscopy image of the microchannel of FIG. 25C and shows the formed chitosan membrane. The images shown in FIGS. 25A-25C have been enhanced to more clearly depict the location of the walls of the microchannel (dotted lines).

FIGS. 26A-26B show the time-dependent growth of membrane thickness. FIG. 26A is a graph showing the time-dependent growth of membrane thickness with chitosan flow rate of 30 μL/min and buffer flow rates of 100-400 μL/min. FIG. 26B is a graph showing the time-dependent growth of membrane thickness with chitosan flow rate of 200 μL/min and buffer flow rates of 10-40 μL/min.

FIG. 27 is a graph showing experimental results for conversion of substrate into product by an enzymatically active chitosan membrane in the microfluidic network.

FIG. 28 shows the structure of alginate used to make an alginate scaffold fabrication of a dual membrane AMM.

FIG. 29 shows the formation of an alginate membrane scaffold on a surface of a chitosan AMM in order to form a dual AMM.

FIG. 30, Panels A-C demonstrate the sequential biofabrication of a micro-sandwich AMM. Panels A-C, provide microscopic (left) and fluorescent (right) views of the forming membranes. Panel A shows the formation and positioning of the “central” chitosan membrane. Panel B shows the formation of the alginate membrane scaffold on a first side (“A”) of the chitosan membrane. Panel C shows the formation of the alginate membrane scaffold on a second side (“B”) of the chitosan membrane. The images shown in FIG. 30, Panels A-C (right side) have been enhanced to more clearly depict the location of the walls of the microchannel (dotted lines).

FIG. 31, Panels A-C shows a fabricated chitosan membrane before (Panel A) and after cell assembly with E. coli cells expressing green fluorescent proteins (GFP) (BL21, GFPUV) on one side of a biofabricated chitosan membrane (Panel B) and additionally of red E. coli cells (BL21, DsRed) assembled onto the other side of the chitosan membrane (Panel C). The images shown in FIG. 31, Panels B-C have been enhanced to more clearly depict the location of the walls of the microchannel (dotted lines).

FIG. 32, Panels A-B show that cells assembled onto an alginate scaffold membrane of a dual AMM retain viability (Panel A) and the signaling response of cells to in vitro stimuli, the signal molecules, autoinducer-2 (AI-2) added in the culture medium (Panel B). Fluorescence intensity showing the production of the red fluorescent proteins inside the cells is measured in arbitrary units (a.u.).

FIG. 33, Panels A-B shows the signaling response of MDAI-2 (DsRed) cells to AI-2 from BL21 (UVGFP) cells after 13 hours (Panel A) and the fluorescent response during 29 hours of experiment (Panel B). Fluorescence intensity is measured in arbitrary units. The image shown in FIG. 33, Panel A has been enhanced to more clearly depict the location of the walls of the microchannel (dotted lines).

In the Figures, microchannel plugs or valves for closing the flow are depicted as cross-haired circles.

### DETAILED DESCRIPTION OF THE INVENTION

The present invention relates to a biofabricated “Active Microfluidic Membrane” (“AMM”) in a microfluidic network of a microfluidic device and a method for the in situ biofabrication of such a microfluidic network. More specifically, the invention relates to devices exhibiting (and methods of) positioning (i.e., erecting, modifying or removing a membrane matrix in situ in a microchannel of a microfluidic network of a microfluidic device. In one embodiment, the matrix comprises chitosan. Chitosan is a linear polysaccharide composed of randomly distributed β-(1→4)-linked D-glucosamine (deacetylated unit) and N-acetyl-D-glucosamine (acetylated unit). In another embodiment, the matrix comprises alginate. Alginate is a linear unbranched polymer containing β-(1→4)-linked D-mannuronic acid (“M”) and α-(1→4)-linked L-guluronic acid (“G”) residues. Chitosan membranes may be formed by varying the pH of the environment to render soluble chitosan insoluble. Alginate membranes may be formed by insolubilizing alginate with Ca⁺².

As used herein the term “Active Microfluidic Membrane” (“AMM”) denotes a microfluidic membrane, which may be impermeable, semi-permeable or freely permeable, that comprises one or more types of functional groups sufficient to permit the immobilization or association of one or more types of bio-species. In some embodiments, the invention provides for Active Microfluidic Membranes formed from only a single such matrix substituent (e.g., chitosan or alginate, etc.). In other embodiments, the invention provides for complex hybrid or dual, or multiple Active Microfluidic Membranes (e.g., membranes that comprise matrices of both chitosan and alginate, etc.). Such two or more matrix substituents may be diffused into one another (e.g., the alginate scaffold may be diffused into the chitosan membrane (or vice versa)) to form a hybrid membrane in which the different substituents are integrated into one another. Alternatively, the different substituents can be layered onto one another to form a dual or multiple membrane. For example, the AMM may be composed of a chitosan membrane layer augmented by an alginate membrane layer on one side, or a chitosan membrane layer sandwiched between two alginate membrane layers, or an alginate membrane layer sandwiched between two chitosan membrane layers. Such microfluidic membranes, and particularly such hybrid, dual or multiple Active Microfluidic Membranes may, for example, be employed to provide alternative rigidity, porosity, chemical resistance, chemical functionality, biological functionality, etc. Dual or hybrid membranes can be formed by, for example, permitting calcium ions to diffuse through a chitosan membrane to thereby insolubilize alginate present on the opposite side of the membrane. Likewise, dual or hybrid membranes can be formed by permitting hydroxyl or hydronium ions to diffuse through an alginate membrane to thereby insolubilize chitosan present on the opposite side of such membrane.

In particular, the use of an AMM composed of such substituents permits one to incorporate (and/or release) one or more bio-specie(s) into (or from) the membrane via functionalization of the reactive groups of the substituent (e.g., functionalization of the amine groups of chitosan, or functionalization of the carboxyl groups of alginate, etc.). As used herein the term “bio-species” includes, for example, a protein (e.g., an enzymatic component (e.g., an enzyme, enzymatic substrate, or an enzymatic co-factor), a hormone, a receptor, an antibody or antigen-binding fragment thereof, a receptor ligand, etc.), a substrate of an enzymatic reaction, a co-factor, a nucleic acid, a microorganism (e.g., a virus, bacteria, cell (including a viable or non-viable mammalian cell and particularly a viable or non-viable human cell, etc.)), or a sub-cellular component thereof (and especially a protein, nucleic acid or a virus).

The invention particularly relates to AMMs that comprise two or more species of cells (including bacterial cells, mammalian cells, and especially human cells). Such AMMs may be employed, for example, as bioreactors (e.g., wherein each cell type mediates a step in a reaction (for example, AI-2, etc.), as model systems to evaluate or identify a signaling pathway or cascade in cell-signaling or cascade signaling studies, as model systems to identify interactions between a drug and a cellular receptor, and as model systems to facilitate new drug discovery.

As used herein, the term “biofabricated” refers to a fabrication process conducted (e.g., erecting, modifying or removing an AMM) using biological materials and mechanisms (Liu, Y. et al. (2010) “Biofabrication to build the biology-device” Biofabrication 2:1-21.

As used herein the term “microfluidic device” refers to device that comprises a support having a “microfluidic network” of one or more microfluid channels, each having cross-sectional dimensions in the range of 1-100 μm, and more preferably in the range of 1-20 μm, or 10-50 μm or 10-100 μm, and lengths of 1-10 millimeters, or even 1-10 centimeters. The cross-sectional geometry of the channels may be circular or elliptical, or may be angular (e.g., having 3, 4, 5, 6, or more sides). Channels of different size, length, or geometry may be employed in the same microfluidic network. Such networks of microchannel(s) may have one, or more than one, microchannel circuit(s), each comprising a microchannel having an input port and an outflow port. Any of a variety of methods may be used to mediate fluid flow in the microchannels of the networks of the present invention, including: air or water pressure, magnetic pumping, peristaltic pumping, capillary diffusion, electrophoresis, photophoresis, thermophoresis, etc. The microchannel(s) of the networks of the devices of the present invention may be discrete (i.e., having no junctions for transfer of fluid or analyte from one microchannel circuit to another), or may be interconnected (e.g., with junctions, valves (one way, two-way, or multi-way), etc. so as to permit communication of fluid from one microchannel circuit to another (see. e.g., U.S. Patent Publication No. 20030196714; U.S. Pat. Nos. 7,232,109; 7,216,671; 7,143,787). The microchannels of the present invention may be either closed (such as a pipe), open (such as a grove or trough), open in part (such as a perforated pipe, or a microchannel that is at one part a pipe and at another part a grove or trough). The microchannels may vary in size, and may have regions that serve as reservoirs, mixing regions, separation regions, etc.

The microfluidic networks of the present invention possess membranes (and in particular, biological membranes (e.g., composed of chitosan, alginate, etc.) that are “positionable” in situ. As used herein, the term “positionable” denotes the capacity to erect a new membrane in a microchannel, the capacity to modify the position or extent of an existing membrane in a microchannel, or to partially or totally remove an existing membrane in a microchannel. In a preferred embodiment, the AMM of the present invention are positionable “in situ.” As used herein, an AMM's is said to be positionable “in situ” if it is capable of being positioned by the manipulation of a reactant (e.g., a salt or ionic species) or reaction condition (e.g., temperature, pH, etc.) without disrupting or opening the microchannel. The invention particularly concerns AMM's that are positionable “in situ” by manipulating the extent of a fluid (or the constituents of such fluid) flowing across or through the membrane.

The microfluidic networks of the present invention may have arbitrary complexity. As used herein the term “arbitrary” complexity is intended to denote that the design, shape, orientation, etc. of the microfluidic network of the device is not constrained or limited, but rather is determined on the discretion of the designer of the network.

According to one embodiment, the disclosed AMM comprises a free standing chitosan membrane fabricated in situ in a microscale fluidic network. The membrane may be fabricated via a natural process from the biopolymer chitosan by instituting a pH gradient (e.g., a gradient from pH 4 to pH 10) at an interface of two laminar flows in microfluidics. The resulting membrane may be semi-permeable to enzymatic substrate/product and allow the fluidic streams to either flow through or flow by the membrane, thereby dramatically enhancing the enzymatic conversion efficiency of the microfluidic reactor. As used herein, the term “semi-permeable” denotes the ability of the membrane to block the flow of certain components (especially certain bio-species) while permitting another component (especially a different bio-species) to traverse the membrane. The resulting chitosan membrane may be augmented with other substituents, such as one or more alginate scaffolds, as described in further detail below.

An in situ generation of pH gradients is exploited as the driving force for membrane assembly in microfluidic devices. Electrochemically-generated pH gradients may be formed by electrical signals in microfluidic channels under flowing conditions for isoelectric focusing (Cabrera, C. R. et al. (2001) “Formation of natural pH gradients in a microfluidic device under flow conditions: model and experimental validation,” Analytical Chemistry, 73(3):658-666) and transverse isoelectric focusing (“IEF”) (Macounova, K. et al. (2001) “Concentration and separation of proteins in microfluidic channels on the basis of transverse IEF,” Analytical Chemistry, 73(7):1627-1633). Non-electrochemical generation of pH or other chemical gradients inside microfluidic networks are demonstrated by converging multiple flow streams within a gradient generator (Dertinger, S. K. W. et al. (2001) “Generation of Gradients Having Complex Shapes Using Microfluidic Networks,” Analytical Chemistry, 73(6):1240-1246; Jeon, N. L. et al. (2002) “Neutrophil chemataxis in linear and complex gradients of interleukin-8 formed in a microfabricated device,” Nature Biotechnology, 20(8):826-830). The generation of gradients with laminar flow systems may be employed in various applications (Zhou, Y et al. (2009) “Generation of complex concentration profiles by partial diffusive mixing in multi-stream laminar flow,” Lab on a Chip, 9:1439-1448; Hattori, K et al. (2009) “Generation of arbitrary monotonic concentration profiles by a serial dilution microfluidic network composed of microchannels with a high fluidic-resistance ratio,” Lab on a Chip, 9:1763-1772; Du, Y et al. (2009) “Rapid generation of spatially and temporally controllable long-range concentration gradients in a microfluidic device,” Lab on a Chip, 9:761-767; Sun, K et al. (2008) “Modular microfluidics for gradient generation,” Lab on a Chip, 8:1536-1543).

Biofabrication exploits biologically-derived materials and biocatalysts for fabrication and offers opportunities to access a wider range of fabrication options (Yi, H. M. et al. (2005) “Biofabrication of Chitosan,” Biomacromolecules, 6(6):2881-2894; Wu, H. C. et al. (2009) “Biofabrication of antibodies and antigens via IgG-binding domain engineered with activatable pentatyrosine pro-tag,” Biotechnology and Bioengineering, 103(2):231-240). Self-assembly (Yi, H. M. et al. (2004) “A robust technique for assembly of nucleic acid hybridization chips based on electrochemically templated chitosan,” Analytical Chemistry, 76(2):365-372), enzymatic assembly (Ulijn, R. V. (2006) “Enzyme-responsive materials: a new class of smart biomaterials,” Journal of Materials Chemistry, 16:2217-2225), and directed assembly (Shi, X. W. et al. (2008) “Chitosan biotinylation and electrodeposition for selective protein assembly,” Macromolecular Bioscience 8:451-457; Lewandowski, A. T. et al. (2008) “Protein assembly onto patterned microfabricated devices through enzymatic activation of fusion pro-tag,” Biotechnology and Bioengineering, 99(3):499-507; Luo X et al. (2008), supra, Lab on a Chip 8:420-430; Park, J. J. et al. (2006) “Chitosan-mediated in situ biomolecule assembly in completely packaged microfluidic devices,” Lab on a Chip, 6:1315-1321) are biofabrication approaches that have been exploited to assemble biological species onto solid surfaces. For example, stimuli-responsive alginate gels may be formed in microfluidic channels (Sugiura, S. et al. (2008) “Tubular gel fabrication and cell encapsulation in laminar flow stream formed by microfabricated nozzle array,” Lab on a Chip, 8:1255-1257; Sugiura, S. et al. (2005) “Size control of calcium alginate beads containing living cells using micro-nozzle array,” Biomaterials, 26(16):3327-3331; Workman, V. L. et al. (2008) “On-chip alginate microencapsulation of functional cells,” Macromolecular Rapid Communications, 29(2):165-170; Zhang, H. et al. (2007) “Exploring microfluidic routes to microgels of biological polymers,” Macromolecular Rapid Communications, 28(5): 527-538).

Chitosan's pH-responsive properties make it uniquely amenable to biofabrication (Yi, H. M. et al. (2005), supra, Biomacromolecules, 6(6):2881-2894; see also U.S. Pat. No. 7,094,372 to Wang et al.). Chitosan is a natural polymer, which is a polysaccharide comprised of [(1,4)-2-amino-2-deoxy-β-D-glucan]. Chitin is also a natural polymer comprised of [(1,4)-2-acetamido-2-deoxy-β-D-glucan]. Chitin is the major constituent of the exoskeleton of insects and crustaceous aquatic animals, and the cell walls of fungus. Chitosan of appropriate purity may be readily produced commercially by the partial deacetylation of chitin.

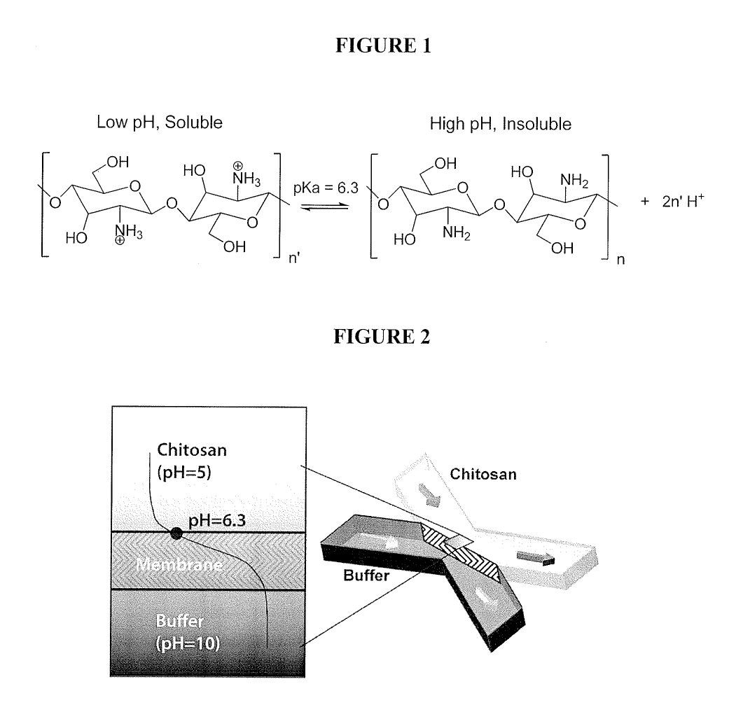

Referring to FIG. 1, chitosan has abundant primary amine groups at the C-2 position of the glucosamine residues, enabling functional properties of chitosan to be exploited for biofabrication (Payne, G. F. et al. (2007) “Chitosan: a soft interconnect for hierarchical assembly of nano-scale components,” Soft Matter, 3:521-527). At low pH, these amines are protonated and positively charged, and chitosan is a water-soluble cationic polyelectrolyte. At higher pH than a pKₐ of about 6.3, chitosan's amines become deprotonated, so that the polymer loses its charge and becomes insoluble and solidifies with gel or film forming characteristics (e.g. a hydrogel matrix). By utilizing the pH-dependent solubility of chitosan combined with electrical signals to control local pH at electrodes, the stimuli-responsive directed assembly of chitosan may be exploited at electrode surfaces in microchips (Yi, H. M. et al. (2004), supra, Analytical Chemistry, 76(2):365-372; Lewandowski, A. T. et al. (2008), supra, Biotechnology and Bioengineering, 99(3):499-507; Wu, L. Q. et al. (2002) “Voltage-Dependent Assembly of the Polysaccharide Chitosan onto an Electrode Surface,” Langmuir, 18(22):8620-8625; Wu, L. Q. et al. (2003) “Spatially Selective Deposition of a Reactive Polysaccharide Layer onto a Patterned Template,” Langmuir, 19(3):519-524; Yi, H. M. et al. (2005) “Patterned Assembly of Genetically Modified Viral Nanotemplates via Nucleic Acid Hybridization,” Nano Letters, 5(10):1931-1936; Yi, H. M. et al. (2005) “Signal-directed sequential assembly of biomolecules on patterned surfaces,” Langmuir, 21(6):2104-2107) and bioMEMS devices (Lewandowski, A. T. et al. (2008), supra, Biotechnology and Bioengineering, 99(3):499-507; Luo X et al. (2008), supra, Lab on a Chip 8:420-430; Park, J. J. et al. (2006), supra, Lab on a Chip, 6:1315-1321; Luo, X. et al. (2008) “Design optimization for bioMEMS studies of enzyme-controlled metabolic pathways,” Biomedical Microdevices, 10(6):899-908).

Referring to FIG. 2, by employing a pH gradient across aperture openings in microfluidic networks, the in situ microfabrication of a freestanding, semi-permeable chitosan membrane is achieved. By utilizing the unique pH-dependent solubility of chitosan, hydrophilic permeable biopolymer membranes may be formed in microfluidic networks by pH gradients generated at the converging interface between a slightly acidic chitosan solution and a slightly basic solution.

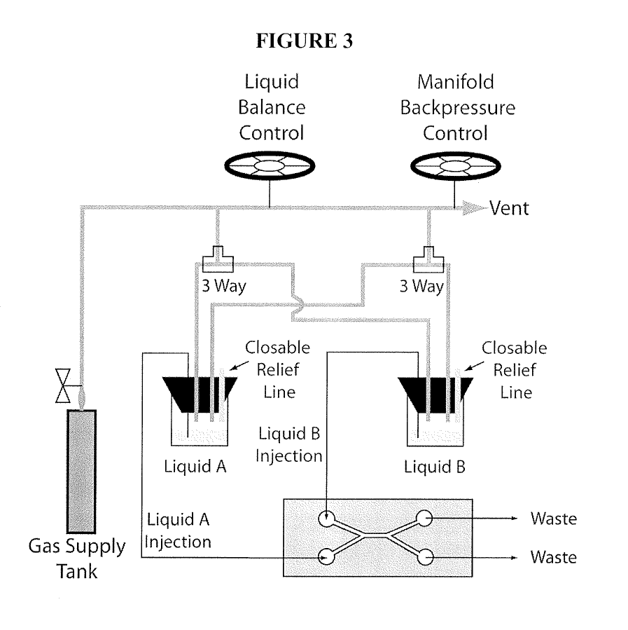

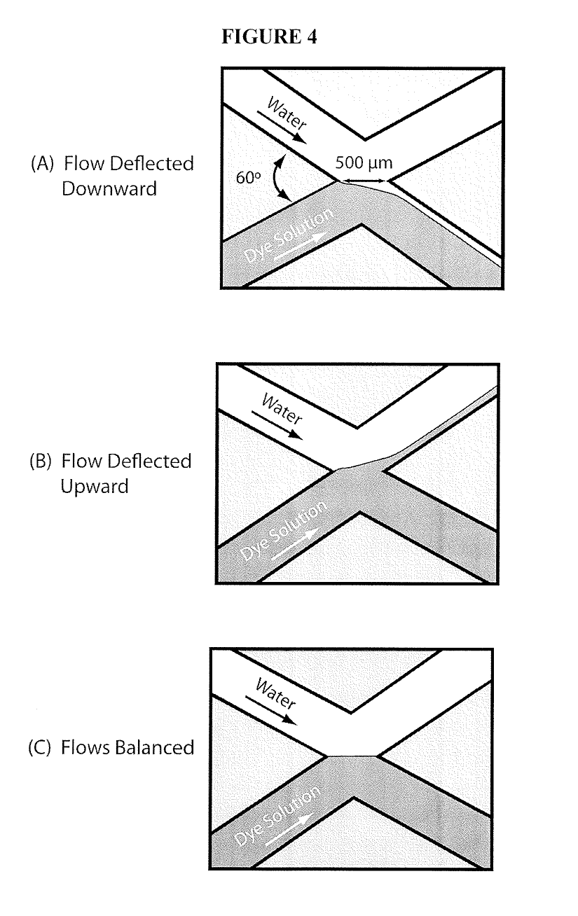

Referring to FIG. 3, an exemplary pneumatic pumping strategy for generating and controlling a stable fluidic interface in a microfluidic network is illustrated. Other pumping strategies may be employed (Braschler, T. et al. (2007) “A simple pneumatic setup for driving microfluidics,” Lab on a Chip 7:420-422). The infrastructure of AMM is the microfluidic network fabricated using a soft lithography technique. In the upstream of the network (e.g. inlet channels), two microchannels converge with a particular angle (e.g. 60°), whereby an interface is maintained between the incoming laminar flows (e.g. water and a dye solution), as shown in FIG. 4.

Preferably, in order to separate individual reaction steps in multi-step biochemical reactions, the downstream configuration of the network (e.g. outlet or waste channels) generally comprises two diverging microchannels. Thus, the fabricated membrane is preferably primarily constrained between the two protruding points of the microfluidic networks, as shown in FIG. 2. The pneumatic pumping manifold allows for fine control of the laminar flow rates and pressures within the channels, so that the flow may be deflected downwards, deflected upwards, or balanced within the channels.

Thus, both a stable flow interface and pH gradient may be realized by incorporating a relatively simple and reliable pumping strategy that exposes the acidic chitosan solution to an adjacent basic buffer. By controlling the interface between the adjacent flow streams of the slightly acidic chitosan solution and a relatively basic solution, a pH gradient is formed at the interface of the laminar flow streams. Further, by tuning the pressure and flow rate of immerging flow streams, the interface of laminar flow streams may be confined in the intersecting microfluidic network.

Chitosan molecules are deprotonated at the interface of the adjacent flow steams, and solidify as a vertical chitosan membrane in a microfluidic device. The thickness or caliper of the resultant biofabricated chitosan membrane is relatively uniform throughout the flow interface, and/or permeable to aqueous solutions, and positionable by mildly acidic solutions. Permeability tests confirm the pore size of the membranes to be a few nanometers, similar to the size of proteins (e.g. antibodies).

The length and height of the formed membrane is determined in part by the geometry of the microchannels. Further, the thickness of the chitosan membrane is partially dependent upon the fabrication process. The more fluid interaction time permitted, the thicker the resulting membrane. The thickness of the membrane is also influenced by the pH of the solutions, as well as the geometry of the microchannels (e.g. chamfered geometry, width, length, etc.).

Because the chitosan membrane is formed by pH gradient, the membrane may be readily erected, modified, dissolved and removed from the microchannel in situ by introduction of a mildly acidic solution. Thus, repeated simple construction and removal of the membrane, or portions of the membrane, may be provided without opening the device or breaking the flow seals. By judicious sequencing of input chitosan fluid, buffer fluid, and acid fluid, and balancing of the formation and dissolution rates, other complex structures may be assembled.

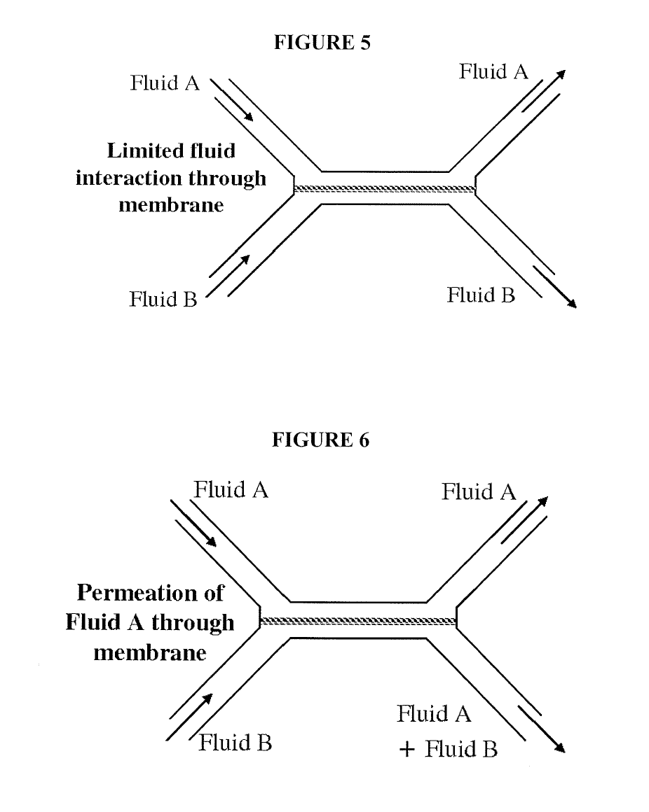

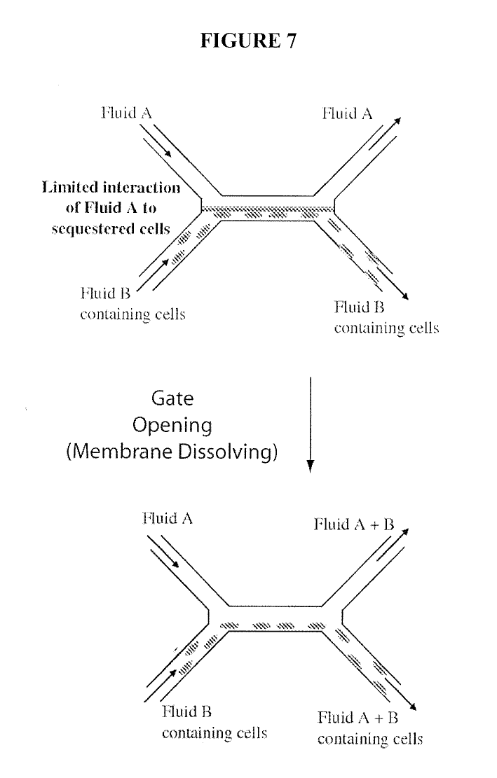

The AMM structure has been found to be permeable to aqueous solutions. The extent of the permeability of the AMM formed in microchannels may be controlled to provide for flow separation, flow permeation, or cell sequestration and gating. As shown in FIG. 5, a chitosan membrane may be formed to temporarily separate different flow streams. As shown in FIG. 6, depending on the properties of the fluids, the membrane may also allow the selected fluids to permeate through the membrane. The membrane may be formed to sequester living cells, as shown in FIG. 7. The membrane may then be dissolved to allow the interaction of the different flow streams or to open a cell chamber to the outer environment.

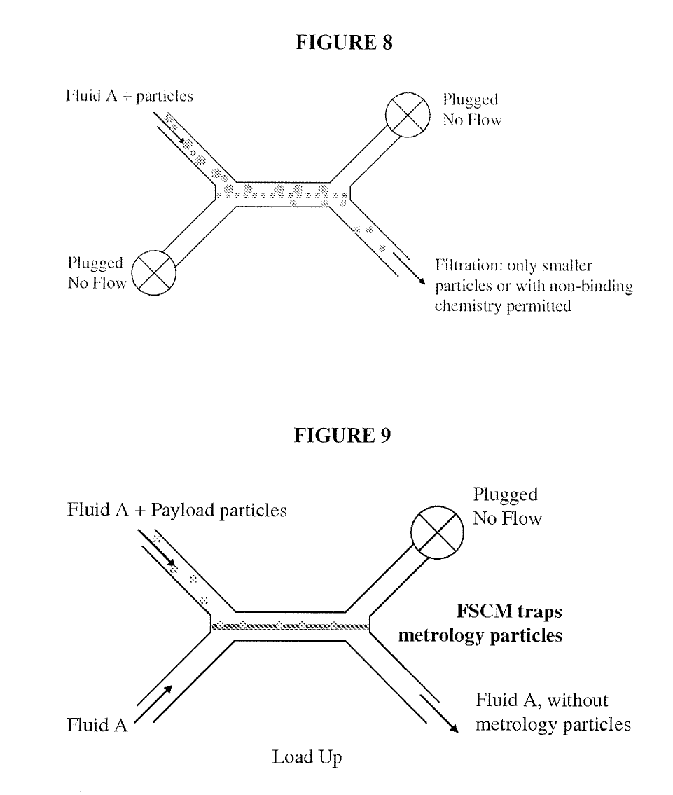

Further, the membrane may be constructed to function as a selective filter, being permeable only to species of a particular size or with non-binding chemistry, as shown in FIG. 8. Alternatively, fluid interaction between the laminar flows may be completely or substantially blocked by the membrane, as noted above.

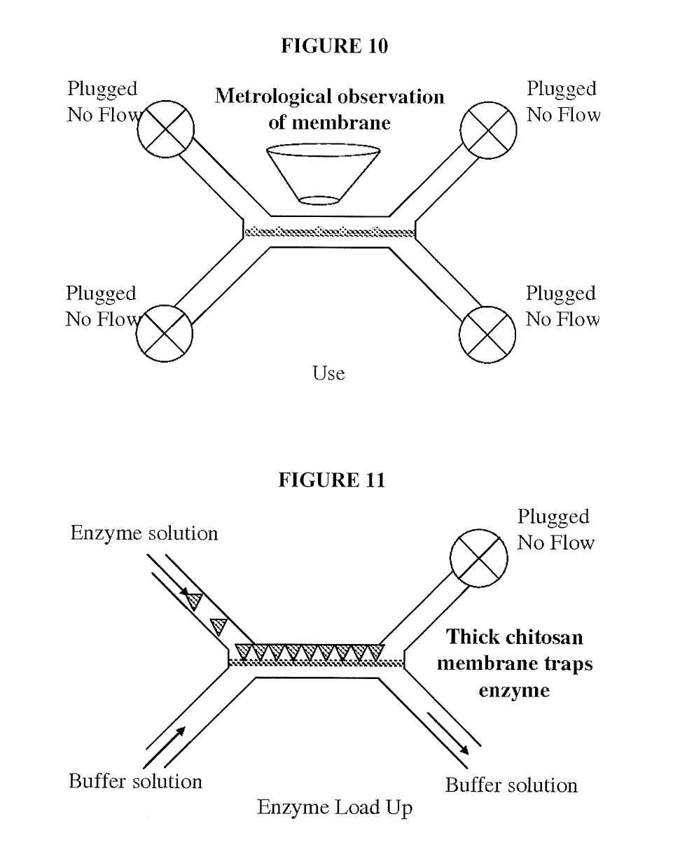

The AMM structures may be functionalized with particles that aid in visualization or measurement, such that metrology applications may be conducted on the surface of the membrane, as shown in FIG. 9 and FIG. 10. For example, an inflow of fluid A with particles is filtered by the membrane. The membrane traps metrology particles, thereby providing an outflow of fluid A without metrology particles, as shown in FIG. 9.

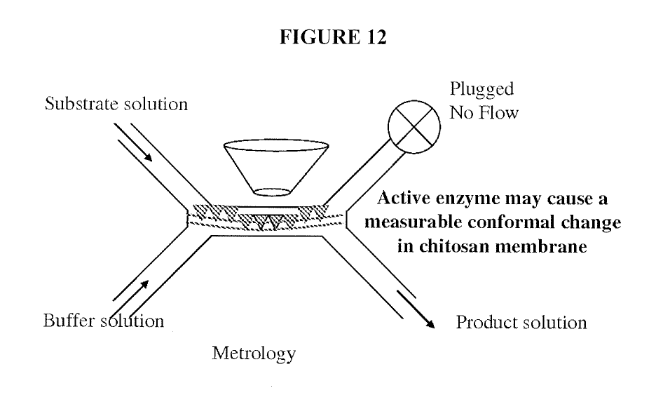

Enzyme-functionalized AMM structures may be incorporated as a biosensor. For example, enzyme may be loaded into the membrane via interaction with an enzyme solution, thus trapping the enzyme within the membrane, as shown in FIG. 11. Increased enzyme bioactivity may affect membrane mechanics, such as causing a measurable conformal change in the membrane, as shown in FIG. 12.

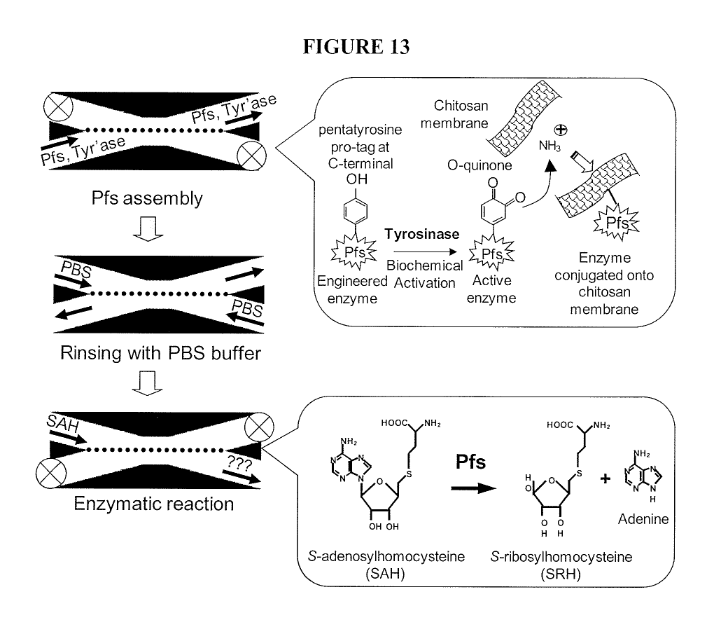

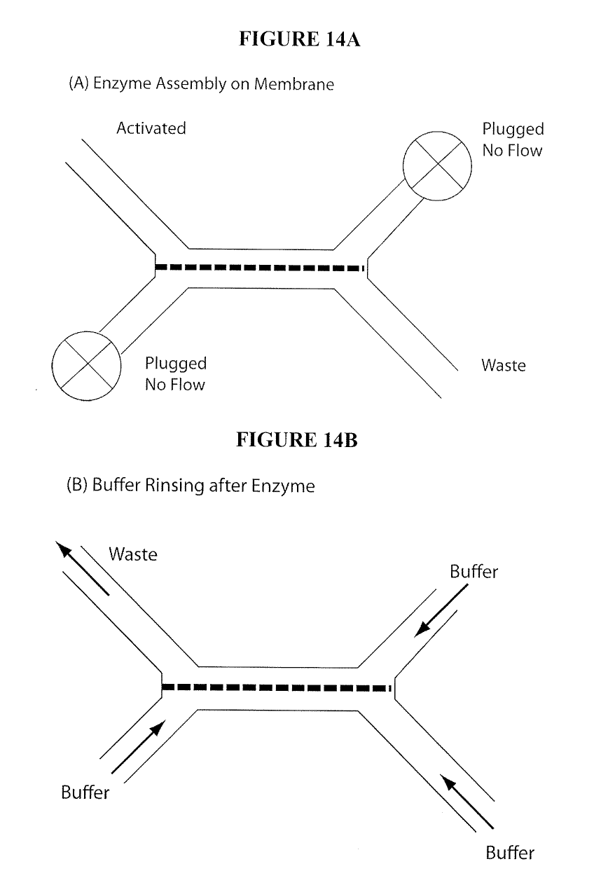

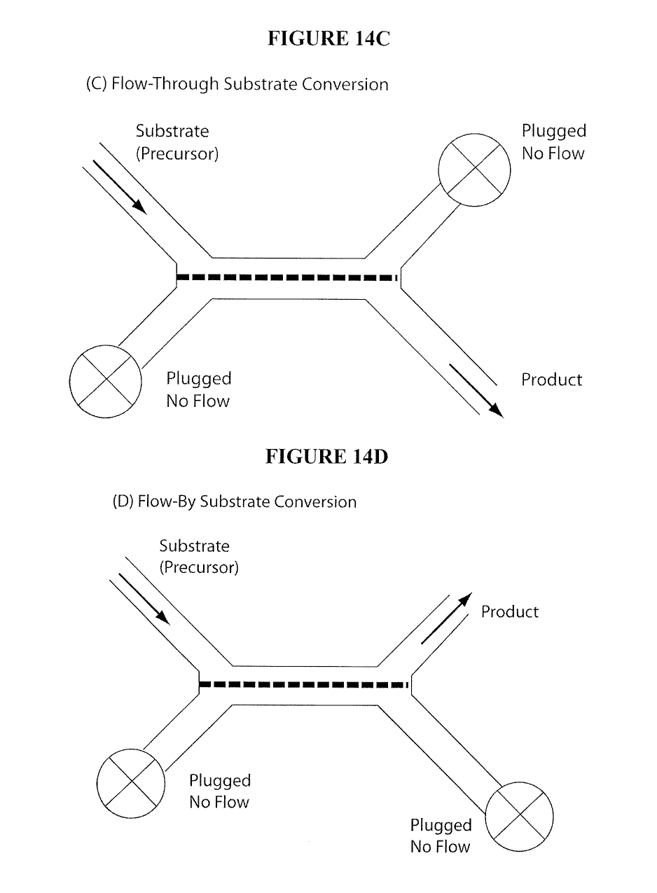

The primary amine groups on chitosan are nucleophilic at neutral state, thus allowing various amine chemistries to be used for covalent conjugation of bio-species such as proteins, nucleic acid (e.g. DNA) and viruses onto chitosan. After the AMM is formed in microchannels, enzyme may be readily immobilized onto the membrane. For example, a metabolic pathway enzyme Pfs (S-adenosylhomocysteine nucleosidase), genetically fused with a pentatyrosine “pro-tag” at its C-terminus, is immobilized on the membrane upon biochemical activation of the pro-tag by tyrosinase (“Tyr'ase”), as shown in FIGS. 13 and 14A. The covalently conjugated enzyme on the membrane withstands the subsequent washing step as buffer flows through and by the chitosan membrane, as shown in FIGS. 13 and 14B. The catalytically active membrane then serves as an enzymatic reaction site as the substrate flows through or flows by the semi-permeable membrane, as shown in FIGS. 14C and 14D. This in situ functionalization converts the membrane into an active element in the microfluidic network.

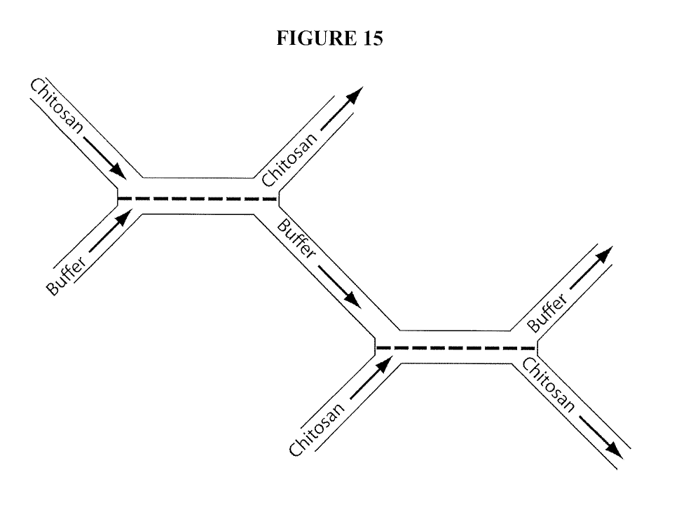

It should be understood that a device may include one or more membranes. For example, two or more membranes may be formed in series within the network of microchannels, as shown in FIG. 15. Further, the membranes may have similar or differing properties and functionalities.

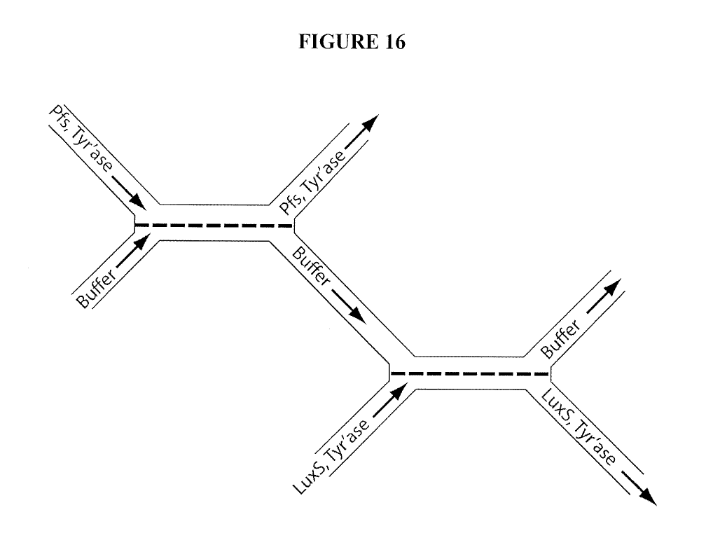

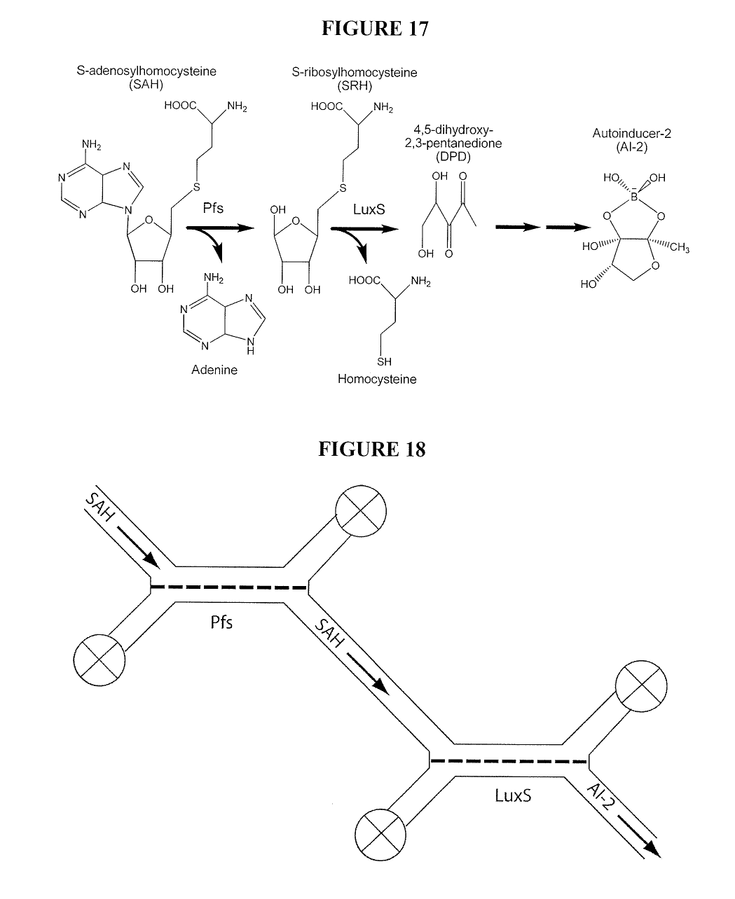

For example, the membrane may be uniquely functionalized with one enzymatic component, or alternatively may be multiply functionalized with more than one enzymatic component on membranes in series, as shown with respect to the chemical reactions shown in FIG. 13 in FIG. 16. FIG. 17 shows the synthesis of Autoinducer-2 (AI-2) from S-adenosylhomocysteine (SAH) via enzymatic reaction of Pfs (S-adenosylhomocysteine nucleosidase) and LuxS (see, Winzer, K. et al. (2002) “LuxS: Its Role In Central Metabolism And The In Vitro Synthesis Of 4-Hydroxy-5-Methyl-3(2H)-Furanone,” Microbiology 148(Pt 4):909-922; Fernandes, R. et al. (February 2009) “AI-2 Biosynthesis Module In A Magnetic Nanofactory Alters Bacterial Response Via Localized Synthesis And Delivery,” Biotechnol. Bioeng. 102(2):390-399. FIG. 18 shows a microfluidic device of the present invention suitable for mediating such synthesis. Functionalization with multiple enzymatic components may be elegantly employed in accordance with the principles of the present invention to reconstruct such or other metabolic pathways in microsystems. For example, the membrane may be functionalized for the immobilization of multiple enzymes on the membrane, for enzymatic reactions in series on catalytic membranes.

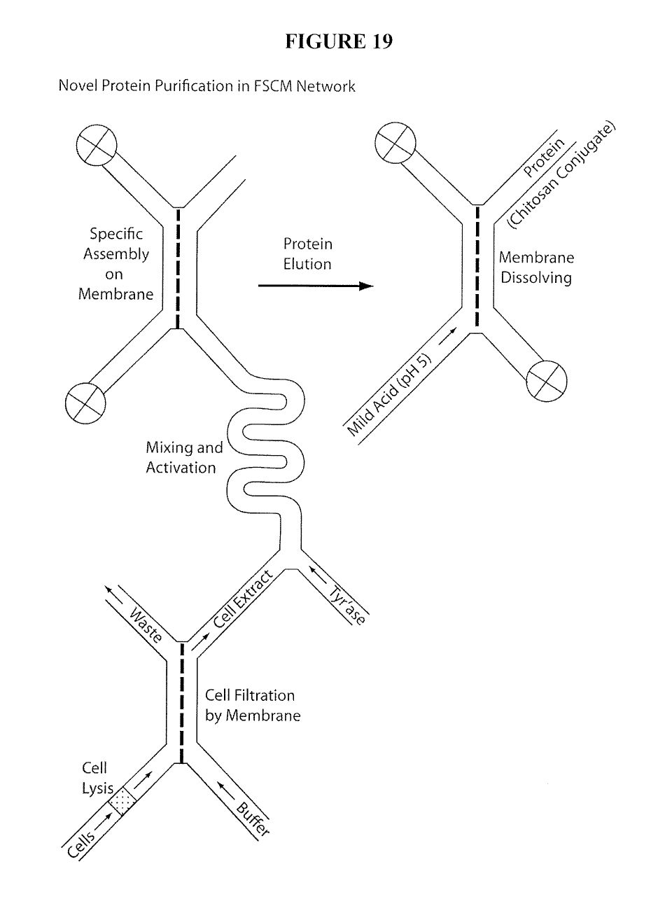

The membrane may be formed at an early stage such that they are embedded with a particular chemistry (“loaded up”), and then dissolved at a later stage to release the chemistry into the downstream flow stream (“payload delivery”). The biopolymer-based membrane structure in microfluidics may thus be a novel vessel for protein purification due to the enzymatic functionality and reversibility of the AMM, as shown in FIG. 19. A target protein is constructed and amplified in the plasmid of bacterial cells, and the lysed and filtered cell extract purified by immobilized metal-ion affinity chromatography (IMAC).

By using the disclosed AMM, the cells may be lysed on chip and filtrated by a chitosan membrane within a microchannel. The soluble cell extract is then mixed with an activation enzyme (e.g., tyrosinase). The target protein with activated pro-tag is immobilized on a second membrane with chemical and spatial specificity. To elute the purified target protein, a mild acid solution (e.g., pH=5) is introduced, which dissolves the membrane structure. The protein-chitosan conjugate can thereby be harvested.

The enzymatic functionality of target protein from the cell extract onto AMM represents multiple steps of routine protein purification in a chromatographic column, storage in a refrigerator, and immobilization onto a spatially patterned area for enzyme assay. The lengthy multi-step processes encountered in conventional chemical engineering techniques are replaced by a one-step functionality onto the AMM in microfluidics.

The eluted target protein-chitosan conjugate has been conferred a pH-responsive property, and is ready for further assay. For example, the protein-chitosan conjugate may be readily assembled onto a patterned inorganic surface in response to applied voltage. The spatially and covalently immobilized protein in the AMM network provides a greatly improved scale-up process compared to conventional processes of patterning in microdevices.

These studies demonstrate the creation of biopolymer membranes using localized pH gradients in microfluidic networks. The membranes may be fabricated in situ and can form networks, gates, and guides. Thus, the disclosed procedure allows for sequential construction of more complex geometries without opening the device, or breaking the seals. By judicious sequencing of input chitosan, buffer and acid fluids, and careful balancing of the formation and dissolution rates, complex structures may be assembled from these building blocks.

The process is biologically benign and relatively simple. The fabricated chitosan membrane “network” may vary with the design of the device, pumping behavior of the solutions into the device, the pH values of the solutions, and other factors, which contribute in part to the final membrane properties such as thickness, permeability and mechanical strength. Thus, the fabrication process may be optimized for specific applications.

Known techniques to assemble biological components including proteins, nucleic acids, viruses and cells onto versatile chitosan scaffolds in microdevices (e.g., see Yi, H. M. et al. (2005), supra, Biomacromolecules, 6(6):2881-2894; Yi, H. M. et al. (2004), supra, Analytical Chemistry, 76(2):365-372; Shi, X. W. et al. (2008), supra, Macromolecular Bioscience,” Macromolecular Bioscience, 8: 451-457; Lewandowski, A. T. et al. (2008), supra, Biotechnology and Bioengineering, 99(3):499-507; Luo X et al. (2008), supra, Lab on a Chip 8:420-430; H Yi, H. M. et al. (2005), supra, Nano Letters, 5(10):1931-1936; Luo, X. et al. (2008), supra, Biomedical Microdevices, 10(6):899-908; Lewandowski, A. T. et al. (2006), supra, Biotechnology and Bioengineering, 93(6):1207-1215) may be extended by the membrane biofabrication processes of the present invention for applications involving membranes within microfluidic devices. Broad applications in metabolic engineering and biosensing may be expanded by assembling active biological components such as biomarkers or enzymes onto the disclosed freestanding chitosan membranes.

Building on the use of chitosan as soft interconnect for biological components (e.g., see Yi, H. M. et al. (2005), supra, Biomacromolecules, 6(6):2881-2894; Yi, H. M. et al. (2004), supra, Analytical Chemistry, 76(2):365-372; Shi, X. W. et al. (2008), supra, Macromolecular Bioscience, 8(5):451-457; Lewandowski, A. T. et al. (2008), supra, Biotechnology and Bioengineering, 99(3):499-507; Luo, X. et al. (2008), supra, Lab on a Chip, 8:420-430; Yi, H. M. et al. (2005), supra, Nano Letters, 5(10):1931-1936; Luo, X. et al. (2008), supra, Biomedical Microdevices, 10(6):899-908; Lewandowski, A. T. et al. (2006) “Tyrosine-based ‘activatable pro-tag’ enzyme-catalyzed protein capture and release,” Biotechnology and Bioengineering, 93(6):1207-1215) and the broad applications of membrane functionalities in microsystems, the rapid in situ biofabrication of freestanding chitosan membranes in microfluidics is relevant to many biochemical, bioanalytical and biosensing applications.

Having now generally described the invention, the same will be more readily understood through reference to the following examples, which are provided by way of illustration and are not intended to be limiting of the present invention unless specified.

### EXAMPLE 1

### Biofabrication of an Active Microfluidic Membrane (AMM) in a Microfluidic Device

### Materials, Preparations and Tests

Chitosan (medium molecular weight, average molecular weight 300,000 g/mol), phosphate buffered saline tablets (10 mM phosphate buffer, 2.7 mM KCl and 137 mM NaCl, pH 7.4), fluorescein (for fluorescence, free acid, λₑₓ=490 nm/λₑₘ=514 nm in 0.1 M Tris pH 8.0) and universal pH indicator (pH 4-10) were purchased from Sigma-Aldrich Corporation, St. Louis, Mo. Sodium hydroxide and hydrochloric acid were purchased from Fisher Scientific, Pittsburgh, Pa. Polydimethylsiloxane (“PDMS”) kits (Sylgard 184 and curing agent) were purchased from Dow Corning, Greensboro, N.C. Microbore PTFE tubing (0.022″ ID/0.042″ OD) was purchased from Cole-Parmer, Vernon Hills, Ill. Genie syringe pumps were purchased from Kent Scientific Corporation, Torrington, Conn. Micro glass slides and single-use syringes were purchased from VWR International, LLC, West Chester, Pa. 5-(and 6-)-Carboxyfluorescein succinimidyl ester (NHS-fluorescein, wavelengths of 495 nm and 519 nm) and FLUOSPHERES® polystyrene nanospheres (20 nm diameter, yellow-green fluorescent (λₑₓ=505 nm/λₑₘ=515 nm, %2 solids) were purchased from Invitrogen Corporation, Carlsbad, Calif., and stored desiccated at −20° C. in a dark container until use. Stainless steel catheter plugs (20 ga×12 mm) were purchased from Instech Solomon, Plymouth Meeting, Pa. A Harris Uni-core punch (1.0 mm) was purchased from Ted Pella Inc., Redding, Calif.

### Chitosan Preparation

A 0.5% chitosan solution was prepared by adding chitosan flakes to de-ionized water, with HCl added dropwise to maintain a pH of about 3, and mixed overnight. The pH was then adjusted to 5 by the dropwise addition of 1 M NaOH. DI water was added to bring the mixture to 0.5%. The resulting chitosan solution was then filtered and stored at 4° C. Fluorescently-labeled chitosan was prepared by reacting NHS-fluorescein with chitosan to produce up to 6% labeled chitosan so that the pH-dependent responsiveness was retained. Details of the labeling procedure are reported in Wu, L. Q. et al. (2003), supra, Langmuir, 19(3):519-524.

### TRITC-Labeled Antibody

Polyclonal rabbit anti Escherichia coli antibody was purchased from AbD Serotec, Oxford UK. Alexa Fluoreporter Texas Red (wavelengths of 577 nm and 603 nm) protein labeling kit was purchased from Invitrogen Corporation, Carlsbad, Calif. TRITC labeling of anti Escherichia coli antibody was performed as per the manufacturer's specification (Invitrogen).

### Microfluidic Device Fabrication

The microchannels were fabricated with PDMS via soft lithography. The angle between the two converging (or dividing) microchannels was either about 30° or about 60°. PDMS microchannels were cured, delaminated from patterned SU-8 molds and punched with input/output holes. For most of the devices, the PDMS microchannels (500 μm wide, 85 μm or 135 μm high) were permanently bonded to piranha-cleaned glass slides by oxygen plasma treatment (450 mTorr pressure, 20 Watts power, 20 sccm oxygen flow rate for 30 seconds) using a Trion RIE machine. For devices used to investigate the membrane microstructure when removal of the membrane was desired, the PDMS microchannels were non-permanently sandwiched between a PDMS layer and a glass slide which, in turn, were compressed between two Plexiglas plates by screws. Liquid flows into the device were instituted via flexible PTFE tubing.

### Pumping Strategy and Membrane Formation

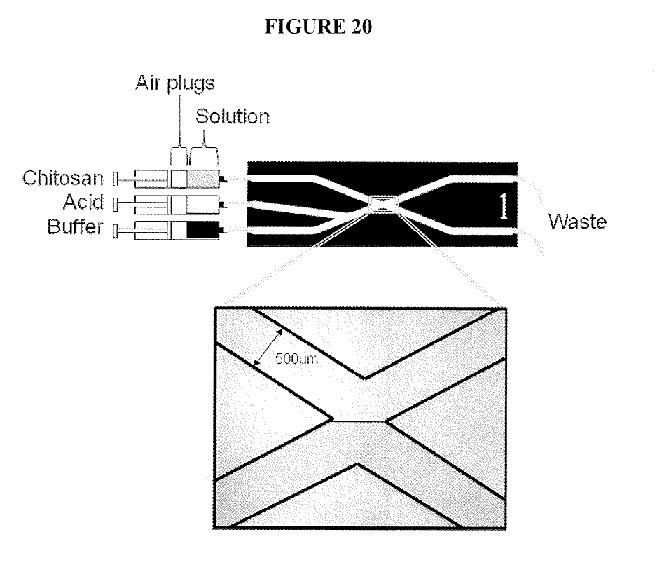

A pumping strategy to produce a stable flow interface and pH gradient is illustrated in FIG. 20. A syringe containing chitosan solution was connected to a fluid input of the fabricated device. Another syringe containing a buffer solution was connected to another fluid input of the device. A third syringe containing an acidic solution was connected to a third fluid input device. The third fluid input was used to introduce the acidic solution to dissolve and remove chitosan membranes for subsequent experimentation. An air plug of about 2 cm³ was introduced into each syringe, with the syringe pumps mounted vertically to position the air plug above the liquid in order to dampen the pulsatile flow often accompanying stepper motors and peristaltic pumps.

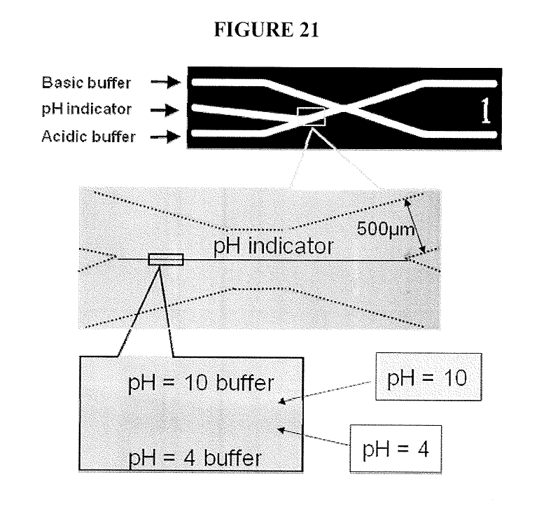

Referring to FIG. 21, a stable, well-balanced flow interface between two dye solutions using the pumping setup was achieved. A stable pH gradient was generated between adjacent flow streams of a basic solution and an acidic solution. The middle flow stream of universal pH indicator (active at pH 4-10) was introduced into the center microchannel, while the basic buffer solution (pH 10) was introduced into the upper-left microchannel and the acidic buffer solution (pH 4) was introduced into the lower-left microchannel.

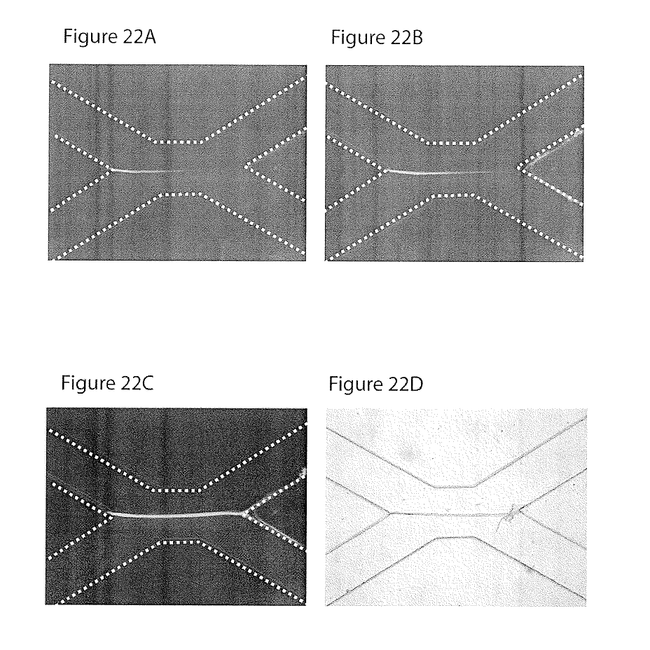

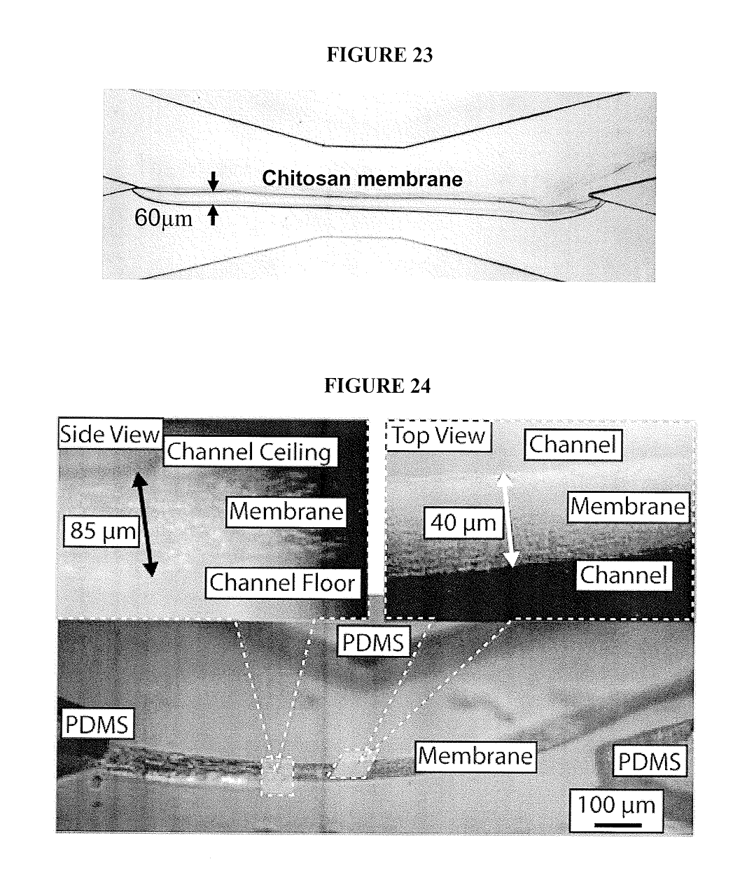

Using this pH gradient, the biofabrication of chitosan membranes at the flow interface was demonstrated, including the biofabrication of straight and T-shaped chitosan membranes. Referring to FIGS. 22A-22D, adjacent flow streams of (1) a NHS fluorescent labeled, slightly acidic chitosan solution, and (2) a relatively basic buffer solution were allowed to contact each other for varied times. Chitosan molecules are deprotonated at the flow interface, causing gelation and solidification of a freestanding vertical chitosan membrane. (The membrane may be referred to as vertical because it is perpendicular to the plane of the microfluidics network). The microstructure of the membrane was relatively uniform throughout the membrane, as shown in FIG. 23.

The PDMS surface at the junction of the two laminar flow streams acts as the nucleation point for membrane growth, where deprotonated chitosan molecules self-assemble onto the PDMS surface. Growth of the free-standing membrane proceeds throughout the flow interface from the upstream nucleation point to the downstream anchoring point where the two laminar streams diverge to the two output channels.

Due to the differences in viscosity of the chitosan solution (0.5% w/v, pH 4.9) and buffer solution (pH 10), the buffer flow rate was much higher than the chitosan flow rate. The chitosan solution was typically set to 10-30 μL/min and the buffer to 100-250 μL/min. For example, the formation of a 1.25 mm-long, 30 μm-thick and 85 μm-high (in terms of microchannel height) chitosan membrane was completed within about 10 minutes. The flow rates used to form this membrane were 10 μL/min for the fluorescent-labeled chitosan and about 160 μL/min for the basic buffer. The angle between the two converging (and dividing) microchannels was about 60°.

Referring to FIG. 24, the microstructure of the chitosan membrane formed by the disclosed approach and within a non-permanently packaged microfluidic device is illustrated. The intact membrane was recovered by filling the microchannels with DI water, freezing the entire device in a −20° C. refrigerator, dissembling the packaging in an ice bath, and then thawing the iced material containing the intact membrane. The membrane on the PDMS was then tilted at a 45° angle for observation under microscope. The membrane was found to be uniform throughout, with a width of about 40 μm and a height of about 85 μm (microchannel height). This structure was similarly obtained in several repeated runs.

### Membrane Microstructure Investigation

To investigate the microstructure of the fabricated membrane, a relatively simple extraction procedure was developed using the non-permanently-sandwiched devices as noted above. After a membrane was fabricated using the non-permanently-bonded device, the microchannels were filled with DI water, disconnected from the syringe pumps and sealed with metal plugs. Next, the microfluidic device was stored at −20° C. in a freezer for 2 hours to encase the membrane in ice. The intact membrane was recovered by first disassembling the whole device in an ice bath and then carefully detaching the PDMS microchannel from the glass slide. The chitosan membrane was thawed in PBS buffer for observation by optical microscopy. This freeze-thaw procedure was adapted from conventional methods, given direct opening of non-permanently sealed microchannels tends to tear the membrane and leave the membrane partially attached to the microchannel ceiling (e.g., PDMS) and/or floor (glass slide).

### Membrane Permeability

The fabricated chitosan membrane was permeable to aqueous solutions and hydroxyl ions. To estimate the pore size of the fabricated chitosan membranes, a series of permeability studies was performed.

Membrane permeability tests were performed by introducing a test solution from one fluid input stream, closing the other fluid input stream, and leaving both fluid outputs open. For all permeability tests, the input solutions were introduced from the lower-left channel, the two right channels were left open, and the upper-left channel was closed. The flow rates were monitored appropriately so that the membrane was not displaced at both ends of the open aperture. The devices used in these studies all had an angle of about 30° between the two converging (dividing) micro channels.

First, a straight membrane of 2.5 mm-long, 60 μm-thick and 135 μm-high (microchannel height) was fabricated. A solution containing small fluorescein molecules (20 μM, molecular size less than 1 nm) was then introduced at 5 μL/min flow rate. The fluorescein solution passed quite freely through the membrane as its presence was noted throughout the fluidic network. Therefore, the fluorescence level was similar in microchannels below and above the membrane.

Next, a solution containing TRITC-labeled antibodies (0.67 μM, molecular size 7-10 nm) was introduced at 5 μL/min flow rate to the same membrane. The antibodies only partially diffused through the membrane while more antibodies were retained at the bottom membrane surface.

A new straight membrane was fabricated in a microchannel. A solution containing FITC-labeled polystyrene nanospheres (2.63×10¹⁴ particles/mL, 20 nm in diameter) was then introduced at 5 μL/min flow rate. The nanospheres were mostly retained at the bottom membrane surface with very few passing through. This was also indicated by the presence of very limited fluorescence in the microchannel above the membrane. The test results for the solution containing FITC-labeled polystyrene nanospheres confirmed the absence of gaps or holes in the assembled membranes.

The permeability studies were repeated three times with new membranes fabricated under substantially identical conditions, and similar results were achieved. Thus, the results from the permeability tests suggest that a representative pore size of the biofabricated chitosan membranes is a few nanometers, similar to the size of antibodies.

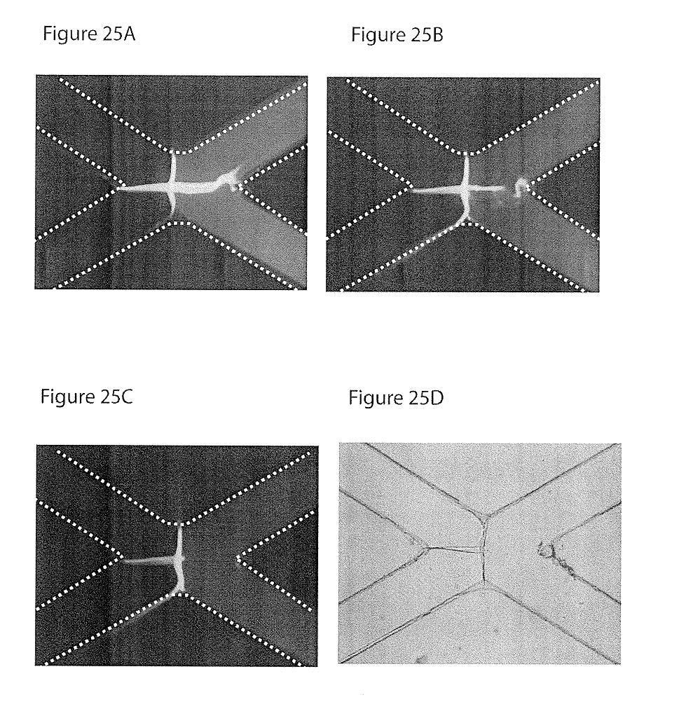

The permeability and manufacturing flexibility of these chitosan membranes was demonstrated by the formation of a T-shaped chitosan membrane inside a microfluidic device, as shown in FIGS. 25A-25D. A horizontal and relatively straight membrane was first formed between the laminar flow paths of the buffer solution and chitosan solution, such as shown in FIG. 24. A portion of the formed horizontal membrane (e.g. the right side portion of the membrane) was then dissolved by a mildly acidic chitosan solution. The remaining portion of the membrane (e.g. the left horizontal side portion of the membrane) was permeable to buffer solution, so that a new vertical membrane could be formed at the interface between the altered laminar flow paths of the buffer solution and the chitosan solution.

In particular, the same chitosan solution was introduced at a reduced flow rate of 3 μL/min from the upper-right channel instead of the lower-left channel. The basic buffer solution was also introduced at a reduced flow rate of 20 μL/min from the upper-right channel, but was exited through the lower-left channel, until the vertical membrane was formed as shown in FIG. 25C. In this way, the two fluids moved from top to bottom and out. The process was monitored under optical microscopy. No displacement of the assembled membrane was observed.

Thus, the right side portion of the original membrane was dissolved by the acidic chitosan solution. The left side of the original membrane was permeable to the basic buffer solution. A new membrane was formed at the new interface between the buffer solution and chitosan solution, and substantially perpendicular to the original membrane.

### Control and Reproducibility of Membrane Thickness

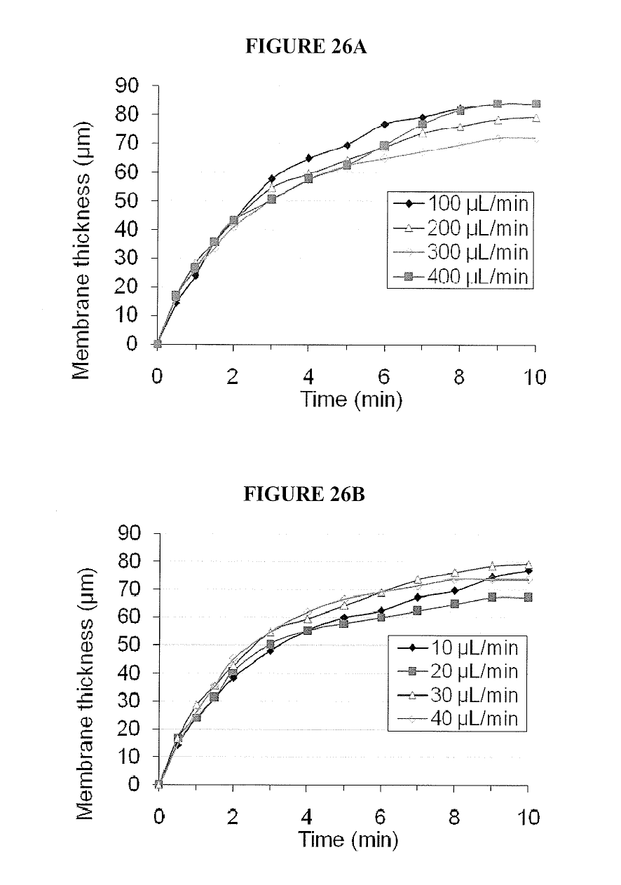

The time-dependent growth of membrane thickness at various chitosan and basic buffer flow rates is depicted graphically in FIGS. 26A and 26B. The chitosan membranes were fabricated by either varying basic buffer flow rates with set chitosan flow rates (30 μL/min, with 100-400 μL/min basic buffer, shown in FIG. 26A), or varying chitosan flow rates with set basic buffer flow rates (200 μL/min, with 10-40 μL/min basic buffer, shown in FIG. 26B). Membrane thickness was monitored by optical microscopy during the 10-min fabrication process.

By careful examination at the assembly process, it was observed that the emerging membrane thickness grew from the interface into the flow stream of chitosan solution over time. This growth was attributed to the diffusion of hydroxyl ions through the fabricated membrane, which caused solidification of chitosan molecules onto the growing membrane while the diffusion of chitosan polymer molecules is likely slower. Interestingly, the results show that the membrane thickness did not change significantly with either chitosan or buffer flow rates. The results demonstrate reproducibility and robustness of the assembly process.

### Enhanced Conversion Efficiency on Catalytic AMM

Other than the surface immobilization of enzyme on the microchannel wall surfaces, where reaction substrate and product are passively transported by diffusion of small molecules to and away from the immobilized enzyme, the semi-permeable AMM with immobilized enzyme allows for the substrate to completely perfuse through the membranes (such as shown in FIG. 14C). The interaction between enzyme and substrate is relatively high, ensuring for a relatively large degree of enzymatic conversion into product.

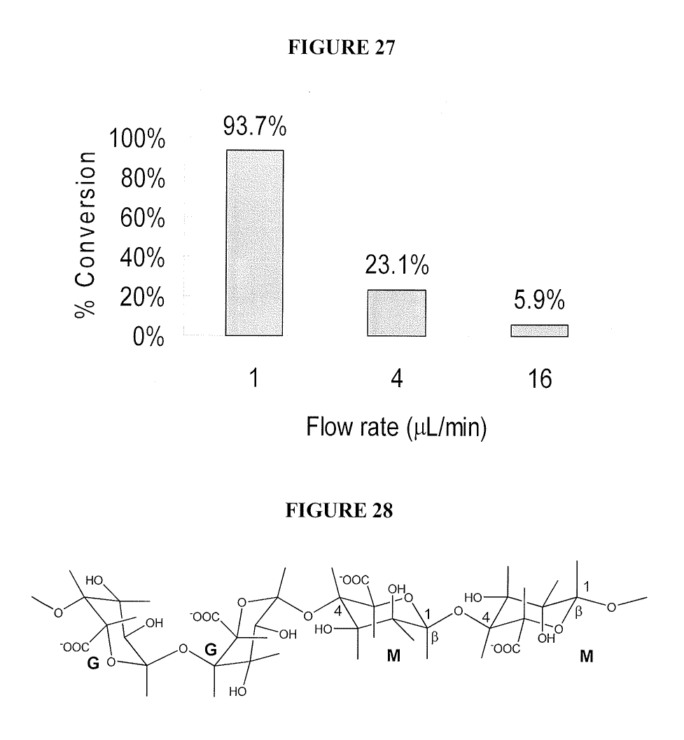

For example, experimental results, depicted graphically in FIG. 27, achieved a 93.7% (flow rate of 1 μL/min) conversion of substrate into product by an enzymatically active chitosan membrane in the microfluidic network. Given the total area of membrane for the experimental design of 0.075 mm², the area-based conversion efficiency has been dramatically enhanced, compared to an approximate 50% conversion efficiency by conventional techniques. By changing the geometry of the microchannel, such as by increasing the microchannel height and length of the channel intersection line, the conversion efficiency of AMM may be further increased. Moreover, scale-up of enzyme assay may be accomplished by parallel operation.

### Discussion

The in situ generation of pH gradients in microfluidic devices for biofabrication of freestanding, semi-permeable chitosan membranes has been demonstrated. The fabricated chitosan membranes, in a range of about 30 μm-thick to about 60 μm-thick, were of uniform cross-section over a relatively long distance (a few mm) at the flow interfaces. Straight and T-shape membranes were employed to demonstrate that they are permeable to aqueous solutions and positionable by mildly acidic solutions. Moreover, these studies demonstrate ease and flexibility of assembling various membrane geometries. Permeability studies suggest that the pore size of the membranes is a few nanometers, similar to the size of antibodies. We believe the facile, rapid biofabrication of freestanding chitosan membranes can be applied to many biochemical, bioanalytical, biosensing applications and cell-based studies.

It is understood that various varieties of chitosan may be employed to form the membrane, including but not limited to, fluorescently labeled chitosan, cross-linked chitosan, and biochemically-conjugated chitosan. Furthermore, the use of the adjective “free standing” to describe the orientation of the membrane is relative. Thus, the orientation of the membrane may be either vertical or horizontal. For example, to achieve a horizontal chitosan membrane, a slightly acidic chitosan flow stream is introduced from above, while a relatively basic solution is introduced from below to join together in the membrane channel to form a horizontally-oriented chitosan membrane, as discussed above.

### EXAMPLE 2

### Biofabrication of a Dual Membrane Active Microfluidic Membrane (AMM) in a Microfluidic Device

3D microenvironments are crucial for in vitro study of cell biology, especially for mammalian cells with limited tolerance to hydrodynamic forces of 2D cell culture systems. In 2D cell culture, cells are displaced as a monolayer on a flat substrate. In 3D cell culture, cells are supported in all directions either by neighboring cells or an extracellular matrix (ECM). Moving from 2D to 3D cell culture systems in microfluidics improves the biological relevance of analyses (Ong, S M et al. (2008) “A gel-free 3D microfluidic cell culture system,” Biomaterials 29(22):3237-3244). Various natural and synthetic hydrogels have been incorporated into microfluidic cell culture systems to support cells in 3D. However, in many cases ultraviolet photo-polymerization and thermo-initiative gelation are cytotoxic to cells (Sundararaghavan, H G et al. (2009) “Neurite growth in 3D collagen gels with gradients of mechanical properties,” Biotechnology and Bioengineering 102(2):632-643 (2009); Tan, W et al. (2003) “Microfluidic patterning of cellular biopolymer matrices for biomimetic 3-D structures,” Biomedical Microdevices 5, (3), 235-244 (2003).

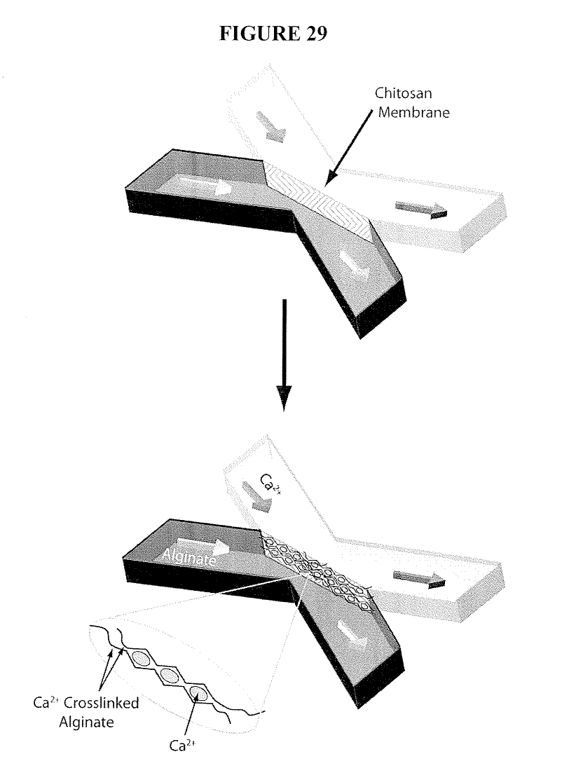

To demonstrate the capabilities and applications of the AMMs of the present invention, a dual membrane AMM was biofabricated to form a 3D micro-sandwich scaffold for cell assembly. The AMM was constructed within a microfluidic device that had been constructed using soft lithography and that comprised microchannels 500 μm wide and 150 μm high. The AMM comprised an initially positioned semi-permeable chitosan membrane that was augmented with an alginate scaffold. The mechanism of alginate scaffold fabrication is schematically shown in FIG. 28. Alginate (1% w/v) was cross-linked into a gel structure along one side of a chitosan membrane by the diffusion of calcium ions (10 mM CaCl₂) through the semi-permeable chitosan membrane, which has pore size of a few nanometers (Luo, X et al. (2008) “Programmable assembly of a metabolic pathway enzyme in a pre-packaged reusable bioMEMS device,” Lab on a Chip 8:420-430). A second alginate scaffold was created on the other side by switching flows, thereby creating a dual or “sandwich” membrane encompassing the original chitosan membrane (i.e., an alginate-chitosan-alginate AMM). A first cell type was introduced into an alginate solution. Upon Ca²⁺-mediated insolubilization of the alginate, the cells became immobilized to an alginate membrane as it was being formed on one side of the chitosan membrane. This process was repeated using the second cell type to thereby immobilize such cells to an alginate membrane as it was being formed on the opposite side of the chitosan membrane. Flow rates of the alginate-cell mixtures were below 3 μL/min to ensure successful seeding. FIG. 29 shows the formation of an alginate membrane scaffold on a surface of a chitosan AMM in order to form a dual AMM.

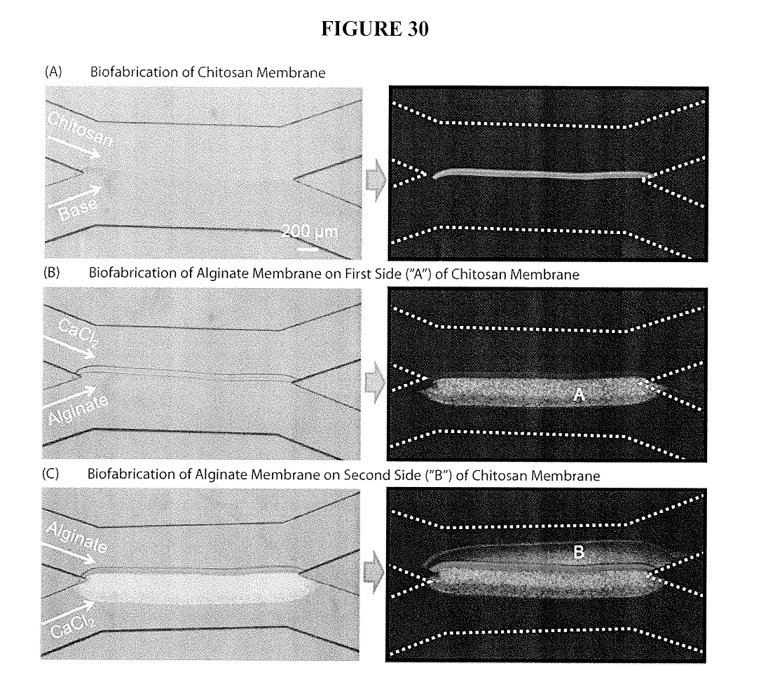

FIG. 30 demonstrates the sequential biofabrication of such a micro-sandwich by first fabricating a freestanding chitosan membrane (labeled with TRITC red fluorescence), followed by fabricating two alginate membranes (decorated with 0.2 μm, green fluorescence-labeled microspheres) on both sides of the pre-fabricated chitosan membrane. The thickness of chitosan and alginate membranes is controllable by setting the time period of the gelation process. The alginate gels attach to the chitosan membrane tightly and they form chitosan/alginate complex due to ionic and electrostatic interactions between the polycations (chitosan) and polyanions (alginate).

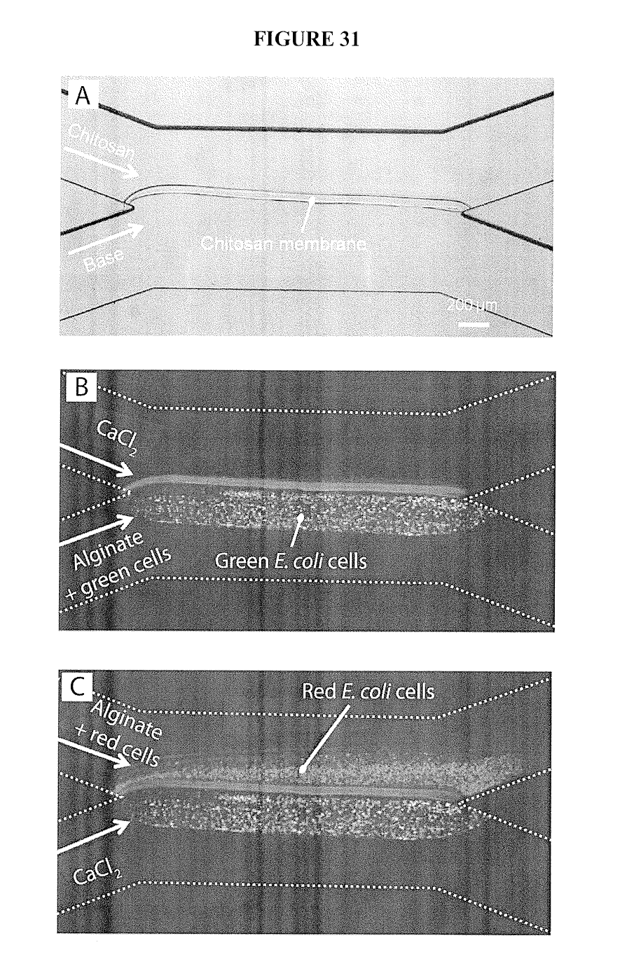

Using the same mechanism, cell assembly was achieved by blending target cells into the alginate solution to embed the target cells in the calcium-crosslinked alginate gel. Alginate gels are commonly used for cell studies in tissue engineering. FIG. 31 shows a fabricated chitosan membrane before (Panel A) and after (Panel B) cell assembly with E. coli cells expressing green fluorescent proteins (GFP) (BL21, GFPUV) on one side of the biofabricated chitosan membrane by calcium gelation with calcium ion diffused from the other side of the chitosan membrane. By switching the inputs for the alginate/cell mixture and the CaCl₂ solution, red E. coli cells (BL21, DsRed) were assembled onto the other side of the chitosan membrane (Panel C) by calcium gelation as well.

The two types of cells were thus assembled in the 3D micro-sandwich scaffolds, with a semi-permeable chitosan membrane providing a supporting backbone for the alginate gels (otherwise alginate gels will easily detach from the hydrophobic PDMS microchannel surfaces) and a physical barrier between the two cell types. Nutrients and signal molecules are thereby free to diffuse through the chitosan membrane for cell growth and cell signaling (as demonstrated by the original Ca² ion diffusion). Importantly, the thickness of the chitosan membrane can be easily tuned with biofabrication time (Luo X L et al. (2010) “In situ generation of pH gradients in microfluidic devices for biofabrication of freestanding, semi-permeable chitosan membranes,” Lab on a Chip 10:59-65), enabling variation in diffusion length between the two alginate membranes. This provides a unique control parameter for cell-to-cell communication studies

In sum, the invention permits the biofabrication of a three dimensional (3D) biopolymer dual membrane Active Microfluidic Membrane, as illustrated by a microfluidic device capable of arraying two cell lines in a micro-sandwich structure. A freestanding chitosan membrane was first fabricated using pH gradients generated at the flow interface of two converging flows. The micro-sandwich was then fabricated by cross-linking alginate on both sides of the chitosan membrane with diffusion of calcium ions through the semi-permeable chitosan membrane. Cell assembly was achieved by blending cells into the alginate solution to embed the target cells into the micro-sandwich alginate scaffolds. The cell assembly process is simple, fast and easy to control.

### EXAMPLE 3

### Viability and Signaling Response of Cells Incorporated into a Dual Membrane Active Microfluidic Membrane (AMM) of a Microfluidic Device

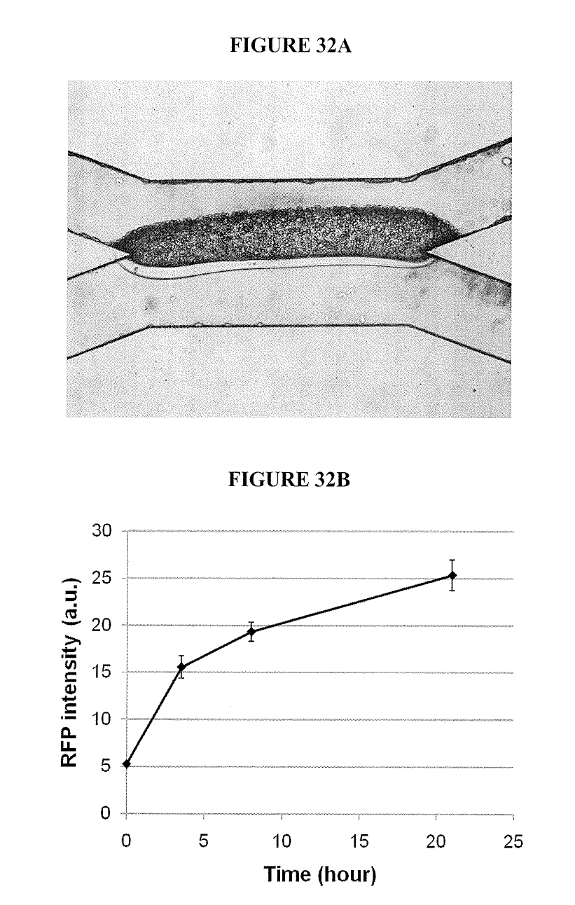

To demonstrate that cells incorporated into an AMM retained viability, red E. coli cells were evaluated in vitro for their ability to fluoresce in response to Autoinducer-2 (AI-2). E. coli BL21 (DsRed) cells were assembled within an alginate scaffold membrane of an alginate-chitosan dual AMM (formed as described above). Luria Both (LB) supplemented with 60 μM AI-2 (signal molecule to stimulate RFP production) and 10 mM CaCl₂ (to maintain alginate gel stability) was introduced into the microchannel at a flow rate of 5 μL/min (in contrast to the 0.2 μL/min flow rate employed for in vivo experiments). FIG. 32A shows that cell density was very high inside the alginate gel after culturing for 5 hours, indicating that the cells remained viable and proliferated dramatically within the alginate scaffold membrane of the dual AMM. As shown in FIG. 32B, the intensity of the red fluorescence protein (RFP) signal was found to increase for the duration of the experiment (21 hours), indicating the continuous signaling response to in vitro AI-2 in LB medium.

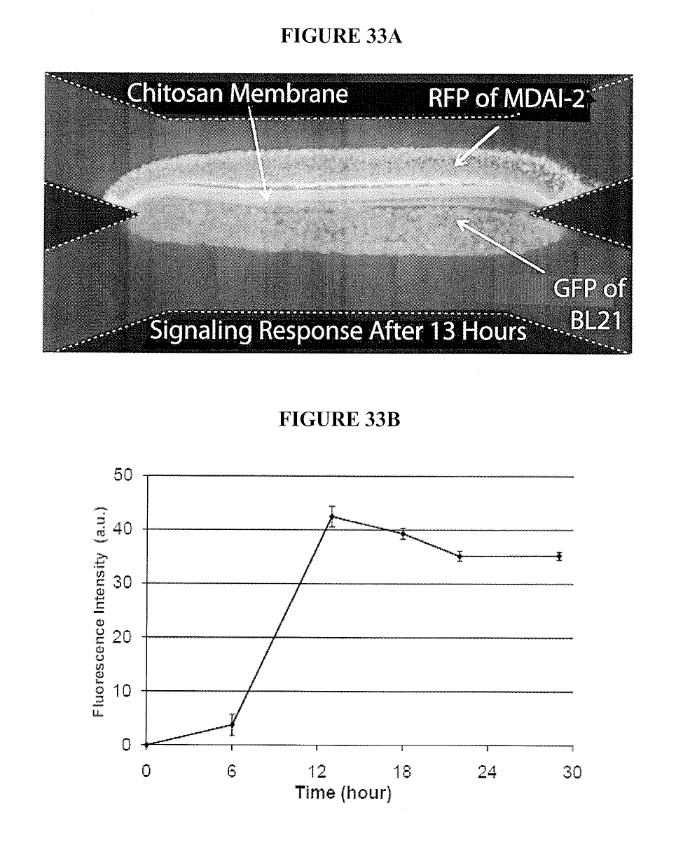

To demonstrate in vivo signaling among different cell lines, E. coli BL21 (GFPUV) and MDAI-2 (DsRed) cells were assembled within the two alginate scaffold membranes of a sandwich AMM (formed as described above). LB supplemented 10 mM CaCl₂ (to maintain alginate gel stability) was introduced into the microchannel at a flow rate of 0.2 μL/min (in contrast to the 5 μL/min flow rate employed for in vitro experiments). FIG. 33A shows the signaling response of MDAI-2 (DsRed) cells to AI-2 from BL21 (UVGFP) cells, which was found to be uniform with a maximum response at around 13 hours (FIG. 33B).

All publications and patents mentioned in this specification are herein incorporated by reference to the same extent as if each individual publication or patent application was specifically and individually indicated to be incorporated by reference in its entirety.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A microfluidic device, comprising:
(A) a support including a microchannel defining a first flow path and a second flow path; and
(B) an Active Microfluidic Membrane (AMM) disposed between the first flow path and the second flow path, the membrane positionable in situ from the microchannel.

2. The device of claim 1, wherein the membrane comprises chitosan.

3. The device of claim 1, wherein the membrane comprises alginate.

4. The device of claim 1, wherein the membrane is semi-permeable and selectively filters a component of one of the first and second flow paths.

5. The device of claim 1, wherein the membrane is permeable to aqueous solutions.

6. The device of claim 1, wherein the membrane is permeable to particles smaller than a given size and impermeable to particles greater than the given size.

7. The device of claim 1, wherein the membrane includes a first portion and a second portion, wherein the second portion is substantially perpendicular to the first portion.

8. The device of claim 1, wherein the microchannel comprises a central portion, and first and second inlet portions in fluid communication with the central portion, the first and second inlet portions converging at the central portion.

9. The device of claim 8, wherein the microchannel further comprises first and second outlet portions in fluid communication with the central portion, the first and second outlet portions diverging from the central portion.

10. The device of claim 1, further comprising a bio-species immobilized on the membrane.

11. The device of claim 10, wherein the bio-species is selected from the group consisting of a protein, a nucleic acid and a virus.

12. The device of claim 10, further comprising an enzymatic component immobilized on the membrane to form a catalytically active membrane serving as an enzymatic reaction site for substrate flowing through or flowing by the membrane.

13. A method of fabricating an Active Microfluidic Membrane (AMM) in a microfluidic device, comprising the steps of:
(A) providing a support defining a sealed microchannel;
(B) generating a fluidic interface between first and second laminar flows within the microchannel; and
(C) fabricating an Active Microfluidic Membrane (AMM) in situ at the fluidic interface.

14. The method of claim 13, wherein the first laminar flow has a first pH and the second laminar flow has a second pH, thereby creating a pH gradient at the fluidic interface during said generating step.

15. The method of claim 14, wherein said fabricating step comprises tuning the pH gradient between the first and second laminar flows.

16. The method of claim 13, wherein said fabricating step comprising fabricating a chitosan membrane.

17. The method of claim 13, wherein the first laminar flow comprises a soluble alginate and the second laminar flow comprises a Ca²⁺ ion during said generating step.

18. The method of claim 17, wherein said fabricating step comprising fabricating an alginate membrane.

19. The method of claim 13, comprising the further step of conjugating a bio-species onto said membrane.

20. The method of claim 19, wherein the bio-species is selected from the group consisting of a protein, a nucleic acid, a virus or a cell.

21. The method of claim 19, wherein said bio-species comprises an enzyme, and wherein said method further comprises the step of enzymatically reacting a substrate flowing through or flowing by the membrane.

22. The method of claim 16, comprising the further step of fabricating an alginate scaffold adjacent the chitosan membrane to form a chitosan/alginate dual Active Microfluidic Membrane (AMM).

23. The method of claim 13, comprising the further step of dissolving in situ at least a portion of the membrane after said fabricating step.

24. The method of claim 23, comprising the further steps of:
(A) maintaining a first membrane portion after said dissolving step;
(B) altering the first and second laminar flows relative to the first membrane portion, thereby generating a secondary fluidic interface between the altered first and second laminar flows; and
(C) fabricating in situ a second membrane portion at the secondary fluidic interface.

25. The method of claim 24, wherein the first membrane portion is angularly disposed relative to the second membrane portion.

26. A method of fabricating in situ a free-standing Active Microfluidic Membrane (AMM) membrane in a sealed microfluidic device by insolubilizing a soluble membrane matrix substituent at an interface of laminar flows within the microfluidic device.

27. The method of claim 26, including the further step of dissolving in situ at least a portion of the fabricated Active Microfluidic Membrane (AMM) using a membrane-solubilizing laminar flow.
